(19) United States
(12) Patent Application Publication
(10) Pub. No.: US 2013/0079423 A1
(43) Pub. Date: Mar. 28, 2013

(54) DIAGNOSTIC METHODS INVOLVING LOSS OF HETEROZYGOSITY

(71) Applicants: Victor Abkevich, Salt Lake City, UT (US); Kirsten Timms, Salt Lake City, UT (US); Alexander Gutin, Salt Lake City, UT (US)

(72) Inventors: Victor Abkevich, Salt Lake City, UT (US); Kirsten Timms, Salt Lake City, UT (US); Alexander Gutin, Salt Lake City, UT (US)

(73) Assignee: Myriad Genetics, Incorporated, Salt Lake City, UT (US)

(21) Appl. No.: 13/581,039

(22) PCT Filed: Feb. 24, 2011
(86) PCT No.: PCT/US2011/026098

§ 371 Date: Dec. 12, 2012

Related U.S. Application Data
(60) Provisional application No. 61/307,761, filed on Feb. 24, 2010.

(51) Int. Cl. C12Q 1/68; G06F 19/00

(52) U.S. Cl. CPC C12Q 1/6886; G06F 19/34
USPC 514/789; 506/9; 435/6.11; 702/19

(57) ABSTRACT

The invention relates generally to methods of molecular analysis and particularly to methods of using genetic copy number variations and loss of heterozygosity in the characterization and treatment of disease.

### FIELD OF THE INVENTION

The invention relates generally to methods of molecular analysis and particularly to methods of using genetic copy number variations and loss of heterozygosity in the detection, characterization and treatment of disease.

### BACKGROUND OF THE INVENTION

Loss of heterozygosity (LOH) is observed when within a chromosomal region one of two copies of the region is lost (i.e., any heterozygous marker within the region loses one of the two alleles). LOH can happen with reduction in copy number, for example, when just one of the two copies is lost. Alternatively, LOH can happen without reduction in copy number, for example, when one of the copies is lost and the remaining copy is duplicated.

LOH is an important feature of many human cancers and can indicate certain characteristics of a patient's particular cancer. Fromont et al., J. UROL. (2003) 170:1394-1397; Valeri et al., UROL. ONCOL. (2005) 23:87-92. Thus, there is a strong need for faster, more sensitive, and more accurate methods of detecting LOH and utilizing LOH information in treating cancer patients.

### BRIEF SUMMARY OF THE INVENTION

It has been discovered that loss of heterozygosity (LOH) in cancer samples can be a powerful indicator of prognosis in cancer patients. More specifically, it has been discovered that LOH in a certain proportion of the genome correlates strongly with shorter overall survival. Thus the present invention generally provides methods of determining cancer patient prognosis using LOH analysis on cancer samples from these patients.

One aspect of the invention provides a method of determining a cancer patient's prognosis comprising determining the amount of overall LOH in a sample containing cancer cells from said patient, wherein high overall LOH indicates a poor prognosis. In some embodiments poor prognosis is an increased likelihood of shorter overall survival. In some embodiments a poor prognosis is indicated if LOH is found in at least a certain percentage (e.g., at least 25%, 30%, 35%, 40%, 45% or more) of the loci analyzed. In some embodiments determining the amount of LOH in a sample comprises isolating nucleic acids from the sample and analyzing the nucleic acid to determine the amount of LOH.

In some embodiments the sample contains no more than a certain level (75%, 70%, 65%, 60%, 55%, 50%, 45%, or 40% or less) of contamination with non-cancerous cells, which can interfere with accurate LOH measurements. In some embodiments the sample is not completely free of contamination, i.e., sample contains at least a certain level (5%, 10%, 15%, 20%, or 25 or more) of contamination with non-cancerous cells. In some embodiments the sample is chosen from a frozen tissue sample and, preferably, a formalin-fixed paraffin embedded (FFPE) sample.

In some embodiments the method further comprises determining LOH for a hotspot (as opposed to a random) locus. In some embodiments the loci analyzed for LOH (particularly the hotspot loci) do not include any of the loci listed in Table A or Table B.

In some embodiments the patient has a particular cancer for which LOH is predictive. Thus in some embodiments the invention provides a method of determining a cancer patient's prognosis comprising determining the amount of LOH in a sample containing cancer cells from said patient, wherein the patient has a cancer chosen from the group consisting of ovarian, breast, lung, prostate and colon, and wherein high LOH indicates a poor prognosis. In some embodiments the cancer is breast cancer. In some embodiments the cancer is ovarian cancer. In some embodiments the cancer is lung cancer. In some embodiments the cancer is prostate cancer. In some embodiments the cancer is colon cancer.

LOH can be observed using different genetic markers, for example, whole genomic sequence, SNPs, microsatellites, short tandem repeats (STRs), etc. Certain techniques and genetic markers allow for determining the genome-wide level of LOH in a sample. For example, whole genome sequencing and/or SNP analysis enable measuring LOH levels at high density across all chromosomes. Thus in some embodiments determining the amount of LOH comprises genome-wide analysis. In some embodiments determining the amount of LOH comprises whole genome sequencing. In some embodiments determining the amount of LOH comprises SNP analysis. In some embodiments at least a certain number (e.g., 5,000) of loci (e.g., SNPs) are analyzed.

Another aspect of the invention provides computer-implemented methods of prognosing patients. Thus the invention provides a computer-implemented method of determining a cancer patient's prognosis comprising:
accessing information on said patient contained in a computer-readable medium;
querying the data stored in said computer-readable medium to obtain LOH information for a sample from said patient;
determining whether said LOH information indicates said sample has high LOH; and
outputting or displaying that said patient has a poor prognosis if said sample has high LOH.

Yet another aspect of the invention provides methods of treatment utilizing LOH. Thus the invention provides a method of treating a cancer patient comprising determining the amount of LOH in a sample containing cancer cells from the patient and administering, prescribing or recommending an aggressive treatment if said sample has high LOH.

Still another aspect of the invention provides a method comprising determining the genome-wide amount of LOH in a patient sample and determining whether a particular prognostic marker has LOH. In some embodiments the loci analyzed for LOH do not include any of the loci listed in Table A or Table B. In some embodiments either a high genome-wide amount of LOH or LOH in the prognostic marker indicates a poor prognosis.

Still another aspect of the invention provides methods (including computer-implemented methods) and systems for accurately determining the copy number of a locus (including LOH) for cancer cells within a sample having significant normal cell contamination.

One aspect of the invention provides a method of determining copy number in a sample comprising applying the analysis outlined in Example 2. In some embodiments of the above invention, the amount of LOH is determined by determining copy number using the analysis outlined in Example 2.

Other features and advantages of the invention will be apparent from the Detailed Description, and from the Claims.

### BRIEF DESCRIPTION OF THE DRAWINGS

FIG. 1 shows LOH frequency across the whole genome in a variety of 15 different cancers (FIG. 1a), 44 breast cancer cell lines (FIG. 1b), and 165 ovarian tumor samples (FIG. 1c).

FIG. 2 shows the distribution of LOH amount over samples (FIG. 2a) as well as the association between LOH amount and survival in ovarian cancer patients (FIGS. 2b & 2c).

FIG. 3 shows the correlation between LOH amount and tumor grade.

FIG. 4 shows a schematic of a computer system according to the present invention.

FIG. 5 illustrates one embodiment of a computer-implemented method of the invention.

FIG. 6 shows the relationship between copy number and signal intensity for high copy number values.

FIG. 7 shows results of copy number analysis of a familial group of samples.

FIG. 8 shows copy number analysis of the ovarian cancer cell line OVCAR8.

FIG. 9 demonstrates the importance of adjustment of copy number analysis based on benign tissue contamination in tumor samples. FIG. 9a shows signal intensities of SNPs for a colon tumor sample. FIG. 9b shows the right copy number solution after adjustment of signal intensity based on the level of contamination with benign tissue. FIG. 9c shows the copy number solution, obtained by the HMM algorithm, if adjustment on contamination with benign tissue is not made.

FIG. 10 illustrates the empirical determination of optimal numbers of loci in methods of the invention.

### DETAILED DESCRIPTION OF THE INVENTION

It has been discovered that loss of heterozygosity (LOH) in cancer samples can be a powerful indicator of prognosis in cancer patients. LOH is observed when one of two heterozygous copies of a chromosomal locus is lost. Any heterozygous marker within the region where one of the two alleles may be lost (e.g., full gene, microsatellite markers, SNPs, etc.) can be used to detect the LOH. LOH can happen with reduction in copy number, for example, when one of the two copies is lost (i.e., the cell only has one total copy left). Alternatively, LOH can happen without reduction in copy number, for example, when one of the copies is lost and the remaining copy is duplicated.

It has been discovered that measuring the level (i.e., rate or amount) of overall LOH in a cancer cell's genome can indicate the aggressiveness of the cancer and thus the patient's prognosis. More specifically, Example 1 shows that LOH in at least a certain number of genome-wide SNP markers correlates strongly with shorter overall survival in cancer patients. Without wishing to be bound by theory, it is thought that a greater level of overall LOH indicates greater overall genetic instability, which in turn indicates a cancer that is more aggressive. Thus the present invention generally provides methods of determining cancer patient prognosis using overall LOH analysis on patient cancer samples.

One aspect of the invention provides a method of classifying cancer in a patient comprising determining the level of overall LOH in a sample containing cancer cells from said patient, wherein high LOH indicates a specific classification. “Loss of heterozygosity” and “LOH” as used herein have their convention meaning in the art and are well understood by those skilled in the art. “Level of LOH” and “amount of LOH” as used herein refer to the number of markers for which LOH has taken place in a particular sample (or in a cell or subset of cells from such a sample). In some embodiments the level of LOH is obtained/expressed as the absolute number of markers for which one allele is lost in a particular cancer sample or cell. In some embodiments the level of LOH in a sample refers to the average amount of LOH over all analyzed cells in the sample. In some embodiments the level of LOH in a sample refers to the average amount of LOH over all cells of a certain type (e.g., cancer cells as opposed to normal cells) in the sample.

“Overall LOH” (e.g., “level of overall LOH”) as used herein refers to an average or aggregate level of LOH across substantially the entire genome of a cell (or group of cells). In particular, “overall LOH” is a measure of the rate of LOH events, regardless of the biological significance of any single event. In this way analyzing “overall LOH” is distinguishable from analyzing LOH at specific hotspot loci. As used herein, a “hotspot locus” means a genomic locus where LOH is associated with some disease or clinical outcome or measure, especially with respect to cancer or some characteristic of cancer (e.g., prognosis). Often loss or amplification of a hotspot locus is known (or thought) to have some biological significance specific to that locus (e.g., LOH at a locus containing a tumor suppressor). Examples of hotspot loci are given in Tables A, B, 8 and 9.

Thus analyzing overall LOH will typically include analyzing a large number of loci that are more or less randomly (or evenly) scattered throughout the genome. In this sense, “random” refers to the fact the loci are not chosen based on any independent biological significance each locus or a group of loci may have. In some embodiments loci may be specifically chosen for analysis and yet still be “random” in this sense, e.g., when the loci are chosen for some superior technical quality (see, e.g., Example 1 below, where the inventors chose the SNPs that were the best and most informative in the assay used). Because the distribution can be random, the markers to be analyzed may incidentally include hotspot loci. In preferred embodiments these hotspot markers make up less than 25%, 20%, 15%, 14%, 13%, 12%, 11%, 10%, 9%, 8%, 7%, 6%, 5%, 4%, 3%, 2%, or 1% of the loci analyzed for LOH in the methods of the invention. In a preferred embodiment no hotspot loci are analyzed.

In some embodiments the level of LOH is expressed as the proportion of LOH (also called “fraction of the genome with LOH” or “FGLOH”), i.e., the proportion of loci showing LOH as compared to the total number of loci analyzed. For example, a level of LOH of 75% can mean that 75% of the markers measured, analyzed and/or yielding reliable results have LOH. When a sample comprising a plurality of cells is being analyzed, this percentage will often be the average proportion of markers having LOH over the relevant portion of the sample analyzed. This approach to calculating the level of LOH is especially useful for determining overall LOH levels, particularly in embodiments of the invention utilizing high-density, genome-wide analysis, where each marker is of roughly equal informative value (e.g., no hotspot loci) and it is essentially the aggregate rate of LOH that is being measured. Such genome screening analysis can, in some embodiments, be paired with more targeted analysis (i.e., using hotspot loci). In some embodiments, the total number of loci analyzed (i.e., those factoring into the calculation of the proportion of LOH) is less than the total number of loci measured by the assay being used (see, e.g., Example 1, where thousands of SNPs were disregarded from the ultimate analysis).

“High LOH” (including high overall LOH) as used herein means the amount of LOH in a patient's sample is greater than some index or reference value (including a threshold index value, as discussed below). In some embodiments the level of LOH (including overall LOH) must be higher than the index value by at least some amount or degree in order to be considered “high LOH.” In some embodiments high LOH means the level of LOH in the sample is at least 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 250-fold, 300-fold, 350-fold, 400-fold, 450-fold, 500-fold, 600-fold, 700-fold, 800-fold, 900-fold, or 1000-fold or more higher than the index value. In some embodiments high LOH means the level of LOH in the sample is at least 25%, 30%, 35%, 40%, 45%, 50%, 55%, 60%, 65%, 70%, 75%, 80%, 85%, 90%, 95% or more higher than the index value. In some embodiments high LOH means the level of LOH in the sample is at least 1, 2, 3, 4, 5, 6, 7, 8, 9, 10 or more standard deviations higher than the index value.

Those skilled in the art will generally appreciate how to obtain and use an index value in the methods of the invention. For example, the index value may represent the amount of LOH found in a normal sample obtained from the patient of interest, in which case an LOH amount in the tumor sample higher than this index value would indicate, e.g., a poor prognosis or increased likelihood of cancer recurrence or a need for aggressive treatment.

Alternatively, the index value may represent the average amount of LOH for a set of individuals from a diverse cancer population or a subset of the population. For example, one may determine the average amount of LOH in a random sampling of patients with cancer (e.g., ovarian, breast, lung, prostate or colon cancer). This average LOH level may be termed the “threshold index value,” with patients having LOH higher than this value expected to have a poorer prognosis than those having LOH lower than this value. In some embodiments the reference population is divided into groups (e.g., terciles, quartiles, quintiles), with each group assigned one or more separate threshold index values (e.g., the average expression level across members of each group, expression levels representing the boundaries of each group, etc.). As shown in Example 1, in some embodiments the threshold index value of FGLOH is 35% (i.e., overall LOH levels higher than 35% are considered “high” and are significantly associated with poor prognosis). In some embodiments the threshold index value of FGLOH is 25%, 30%, 35%, 40%, 45%, 50%, 55%, 60%, 65%, 70%, 75%, 80%, 85%, 90%, 95% or more. As further shown in Example 1, in some embodiments a threshold index value of FGLOH is used to determine a tumor's stage, which in turn may indicate prognosis (e.g., for grade 1 tumors a threshold index value of FGLOH may be 5.6%, for grade 2 tumors a threshold index value of FGLOH may be 23.7%, while for grade 3 tumors a threshold index value of FGLOH may be 73.9%).

Such threshold index values can be determined thusly: In order to assign patients to risk groups, a threshold value will be set for LOH amount. The optimal threshold value is selected based on the receiver operating characteristic (ROC) curve, which plots sensitivity vs (1−specificity). For each increment of LOH amount, the sensitivity and specificity of the test is calculated using that value as a threshold. The actual threshold will be the value that optimizes these metrics according to the artisan's requirements (e.g., what degree of sensitivity or specificity is desired, etc.). Example 1 demonstrates that a high amount of LOH is correlated with poor prognosis and Example 4 demonstrates determination of a threshold value determined and validated experimentally. Thus high LOH can mean the determined amount of LOH is higher than the threshold index value (and it can then be concluded that the patient has an increased likelihood of a poor prognosis, e.g., a shorter overall survival).

Alternatively the index value may represent the average amount of LOH in a plurality of training patients (e.g., breast cancer patients) with similar outcomes whose clinical and follow-up data are available and sufficient to define and categorize the patients by disease outcome, e.g., prognosis. See, e.g., Examples, infra. For example, a “good prognosis index value” of overall LOH can be generated from a plurality of training cancer patients characterized as having “good outcome”, e.g., those who showed overall survival of more than a certain number of months or years, those who have not had cancer recurrence five years (or ten years or more) after initial treatment, or those who have not had progression in their cancer five years (or ten years or more) after initial diagnosis. A “poor prognosis index value” of overall LOH can be generated from a plurality of training cancer patients defined as having “poor outcome”, e.g., those who did not survive more than a certain number of months or years, those who have had cancer recurrence within five years (or ten years, etc.) after initial treatment, or those who have had progression in their cancer within five years (or ten years, etc.) after initial diagnosis. Thus, a good prognosis index value may represent the average level of LOH in patients having a “good outcome,” whereas a poor prognosis index value may represent the average level of LOH in patients having a “poor outcome.”

Example 1 shows that a high amount of overall LOH is correlated with poor prognosis. Thus high overall LOH can mean the determined amount of overall LOH is more similar to the poor prognosis index value than to the good prognosis index value (and it can be concluded that the patient is more likely to have a poor prognosis, e.g., a shorter overall survival). On the other hand, when the determined level of overall LOH is closer to the good prognosis index value than to the poor prognosis index value, then it can be concluded that the patient is more likely to have a good prognosis, i.e., a low (or no increased) likelihood of cancer recurrence.

As used herein, “classifying a cancer” and “cancer classification” refer to determining one or more clinically-relevant features of a cancer and/or determining a particular prognosis of a patient having said cancer. Thus “classifying a cancer” includes, but is not limited to: (i) evaluating metastatic potential, potential to metastasize to specific organs, risk of recurrence, and/or course of the tumor; (ii) evaluating tumor stage; (iii) determining patient prognosis in the absence of treatment of the cancer; (iv) determining likelihood or likely degree of patient response (e.g., tumor shrinkage, overall survival or progression-free survival) to treatment (e.g., chemotherapy, radiation therapy, surgery to excise tumor, etc.); (v) diagnosis of actual patient response to current and/or past treatment; (vi) determining a preferred course of treatment for the patient; (vii) determining likelihood of patient relapse after treatment (either treatment in general or some particular treatment); (viii) prognosis for patient life expectancy (e.g., prognosis for overall survival), etc; all of which are “specific classifications.”

A “negative classification” means an unfavorable clinical feature of the cancer (e.g., a poor prognosis). Examples include (i) an increased metastatic potential, potential to metastasize to specific organs, and/or risk of recurrence; (ii) an advanced tumor stage; (iii) a poor patient prognosis in the absence of treatment of the cancer; (iv) a poor prognosis of patient response (e.g., tumor shrinkage or progression-free survival) to a particular treatment (e.g., chemotherapy, radiation therapy, surgery to excise tumor, etc.); (v) a poor prognosis for patient relapse after treatment (either treatment in general or some particular treatment); (vi) a poor prognosis of patient life expectancy (e.g., prognosis for overall survival), etc. In some embodiments high LOH indicates a negative classification in cancer (e.g., increased likelihood of shorter overall survival).

As used herein, a patient has an “increased likelihood” of some clinical feature or outcome (e.g., shorter survival) if the probability of the patient having the feature or outcome exceeds some reference probability or value. The reference probability may be the probability of the feature or outcome across the general relevant patient population. For example, if (1) the probability of recurrence (or any other clinical feature or outcome) in the general breast cancer population is X %, (2) a particular patient has been determined by the methods of the present invention to have a probability of recurrence of Y %, and (3) if Y>X, then the patient has an “increased likelihood” of recurrence. In some embodiments the patient has an “increased likelihood” of the clinical feature or outcome if the patient's probability of the feature or outcome exceeds the reference probability (e.g., that of the general patient population) by at least some minimum amount (e.g., at least at least 25%, 30%, 35%, 40%, 45%, 50%, 55%, 60%, 65%, 70%, 75%, 80%, 85%, 90%, 95% or more greater than the reference probability; at least 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 250-fold, 300-fold, 350-fold, 400-fold, 450-fold, 500-fold, 600-fold, 700-fold, 800-fold, 900-fold, or 1000-fold or more greater; at least 1, 2, 3, 4, 5, 6, 7, 8, 9, 10 or more standard deviations greater). Alternatively, as discussed above, a threshold or reference value may be determined and a particular patient's probability of recurrence may be compared to that threshold or reference. Because predicting recurrence and predicting progression are prognostic endeavors, “predicting prognosis” will often be used herein to refer to either or both. In these cases, a “poor prognosis” will generally include to an increased likelihood of recurrence, progression, or both.

As discussed above, it has been determined that high LOH is strongly associated with poor prognosis. It is thought that high LOH accompanies more aggressive cancer cells. Such a cancer in a patient will often mean the patient has an increased likelihood of shorter survival (e.g., due to recurrence or progression). This can of course be stated differently, for example, the patient has a decreased likelihood of longer survival or simply the patient has a poor prognosis for survival. Such a cancer in a patient can also mean the patient has an increased likelihood of recurrence after treatment (e.g., the cancer cells not killed or removed by the treatment will quickly grow back). Such a cancer can also mean the patient has an increased likelihood of cancer progression or more rapid progression (e.g., the rapidly proliferating cells will cause any tumor to grow quickly, gain in virulence, and/or metastasize). Such a cancer can also mean the patient may require a relatively more aggressive treatment.

Thus, in some embodiments the invention provides a method of classifying cancer comprising determining the amount of overall LOH in a sample obtained from a patient, wherein high overall LOH indicates an increased likelihood of shorter survival. In some embodiments the invention provides a method of classifying cancer comprising determining the amount of overall LOH in a sample obtained from a patient, wherein high overall LOH indicates an increased likelihood of recurrence or progression. In some embodiments the invention provides a method of classifying cancer comprising determining the amount of overall LOH in a sample obtained from a patient, wherein high overall LOH indicates the patient may require a relatively more aggressive treatment.

In some embodiments high LOH (and a poor prognosis) is indicated if LOH is found in at least a certain percentage of the markers or loci (e.g., SNPs, base pairs, etc.) analyzed. In some embodiments high LOH means LOH in at least 25%, 30%, 35%, 40%, 45%, 50%, 55%, 60%, 65%, 70%, 75%, 80%, 85%, 90%, 95%, 96%, 97%, 98%, 99%, or 100% of the markers or loci analyzed (recall that the loci analyzed need not be all of the loci measured in the assay). In some embodiments high LOH means the number of markers or loci with LOH in the test sample is at least 2×, 3×, 4×, 5×, 6×, 7×, 8×, 9×, 10×, 15×, 20×, 25×, 30×, 35×, 40×, 45×, 50×, 60×, 70×, 80×, 90×, 100×, 150×, 200×, 250×, 300×, 350×, 400×, 450×, 500×, 600×, 700×, 800×, 900×, or 1000× or more of the reference number of markers or loci with LOH (e.g., loci with LOH in a reference sample, the average index value of LOH in a subject population, etc.).

As discussed above, this amount of LOH may represent the average LOH in all cells of a sample, a certain subset of cells from the sample, a single cell, etc. In some embodiments the amount of LOH for a sample is the average amount of LOH in at least 10%, 15%, 20%, 25%, 30%, 35%, 40%, 45%, 50%, 55%, 60%, 65%, 70%, 75%, 80%, 85%, 90%, 95% or more of the cells in the sample. In some embodiments the amount of LOH for a sample is the average amount of LOH found in at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the markers tested.

In some embodiments at least a certain number of markers or loci (e.g., base pairs, SNPs, etc.) are analyzed. In some embodiments at least 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, 900,000, 1,000,000, 1,250,000, 1,500,000, 1,750,000, 2,000,000, or more markers or loci are analyzed for LOH.

LOH can be measured using various techniques and different genetic markers, for example, whole genomic sequencing, SNPs, microsatellites, short tandem repeats (STRs), etc. Genome-wide analysis is particularly useful according to the present invention because this can detect overall LOH across all chromosomes and give a measure of the aggregate rate or amount of LOH (which can in turn indicate the overall genetic instability and aggressiveness of the tumor cell). Genome-wide analysis need not analyze every relevant locus (e.g., base pair, SNP, etc.) in the genome. Instead genome-wide analysis refers to analyzing a large number of loci ( ) more or less randomly spaced within the genome. Thus in some embodiments determining the amount of overall LOH comprises genome-wide analysis. Such genome-wide analysis includes, but is not limited to, whole genome sequencing and genome-wide SNP analysis. Thus in some embodiments determining the amount of overall LOH comprises whole genome sequencing. In some embodiments determining the amount of overall LOH comprises genome-wide SNP analysis. Any technique capable of determining genotypes at particular SNPs may be used for such SNP analysis (e.g., sequencing or microarray, as discussed in more detail below). Genome-wide analysis will often involve testing numerous markers randomly spaced throughout the genome, rather than testing markers independently predictive of prognosis (i.e., hotspot loci). In some embodiments these loci are randomly placed along the genome. In some embodiments these loci are known SNPs. In some embodiments LOH analysis involves amplification of one or more nucleic acids (e.g., whole genome DNA by a whole genome amplification method). In some embodiments the whole genome amplification method uses a strand displacing polymerase and random primers

Various types of samples may be used in the different aspects and embodiments of the invention. Indeed, any sample from which LOH information can be gleaned may be used. As will be apparent to a skilled artisan apprised of the present invention and the disclosure herein, “tumor sample” means any biological sample containing one or more tumor cells, or one or more tumor derived RNA or protein, and obtained from a cancer patient. For example, a tissue sample obtained from a tumor tissue of a cancer patient is a useful tumor sample in the present invention. The tissue sample can be an FFPE sample, or fresh frozen sample, and will preferably contain largely tumor cells. A single malignant cell from a cancer patient's tumor is also a useful tumor sample. Such a malignant cell can be obtained directly from the patient's tumor, or purified from the patient's bodily fluid or waste such as blood, urine, or feces. In addition, a bodily sample such as blood, urine, sputum, saliva, or feces containing one or tumor cells, or tumor-derived RNA or proteins, can also be useful as a tumor sample for purposes of practicing the present invention.

The inventors have discovered (Example 2, especially Tables 8 & 9), and there are known in the art, LOH hotspot loci. For example, it has been shown that LOH at specific chromosomal locations can be used to predict cancer aggressiveness. Franko et al., J. GASTROINTEST. SURG. (2008) 90:1664-1672. The inventors have found additional regions of deletion and amplification in ovarian cancer (Example 2 below). Thus in one aspect the invention provides a method of classifying cancer comprising determining in a patient sample whether a genomic region listed in Table 8 or Table 9 (or a gene contained therein) has LOH, wherein LOH in the genomic region (or gene) indicates a poor prognosis. Thus in one aspect the invention provides a method of classifying cancer comprising determining in a patient sample the copy number of a gene or genomic region listed in Table 8 or Table 9, wherein a copy number below 2 for a gene or genomic region listed in Table 8 or a copy number above 2 for a gene or genomic region listed in Table 9 indicates a poor prognosis. In some embodiments a copy number above 3, 4, 5, 6, 7, 8, 9, 10 or more for the gene or genomic region listed in Table 9 indicates a poor prognosis. In some embodiments LOH in the gene or genomic region listed in Table 8 indicates a poor prognosis.

In one aspect of the invention genome-wide LOH analysis is paired with more targeted analysis of specific marker(s). For example, in breast cancer HER2 status can indicate a particular clinical subtype that will respond to a particular drug. Thus determining the amount of LOH can indicate aggressive cancer and determining HER2 status can indicate possible treatments. In some embodiments overall LOH analysis is paired with analysis of LOH hotspots. Examples of LOH hotspots include those known in the art as well as the genes and genomic regions listed in Table 8. In some embodiments the loci (including hotspots) analyzed do not include those listed in Table A and Table B. In some embodiments the cancer is ovarian cancer and the loci analyzed do not include those listed in Table A. In some embodiments the cancer is prostate cancer and the loci analyzed do not include those listed in Table B.

TABLE A

| | Chromosomal | |
|---|---|---|
| Locus | Location | Reference |
| D11S2071 | 11p15.5-15.3 | Lu et al., CANCER RES. (1997) 57: 387-390 |
| D11S988 | 11p15.5-15.3 | Lu et al., CANCER RES. (1997) 57: 387-390 |
| D6S473 | 6q25.1-25.2 | Colitti et al., ONCOGENE (1998) 16: 555-559 |
| TEL | 12p12.3 | Hatta et al., BR. J. CANCER (1997) 75: 1256-1262 |
| NF1 | 17q11.2 | Wertheim et al., ONCOGENE (1996) 12: 2147-2153 |
| D7S523 | 7q31 | Koike et al., GENES CHROMOSOMES CANCER (1997) 19: 1-5 |
| D5S644 | 5q14-21 | Tavassoli et al., BR. J. CANCER (1996) 74: 115-119 |
| D6S300 | 6q14 | Jiang et al., CANCER RES. (1996) 56: 3534-3539 |
| D9S144 | 9q23-22 | Rodabaugh et al., CANCER RES. (1995) 55: 2169-2172 |
| GSN | 9q32-34 | Devlin et al., BR. J. CANCER (1996) 73: 420-423 |
| D14S267 | 14q32 | Bandera et al., CANCER RES. (1997) 57: 513-515 |
| D14S80 | 14q12-13 | Bandera et al., CANCER RES. (1997) 57: 513-515 |
| AR | Xq11-12 | Cheng et al., J. NATL. CANCER INST. (1996) 88: 510-518 |
| D9S59 | 9q31-33 | Devlin et al., BR. J. CANCER (1996) 73: 420-423 |
| D6S284 | 6q14-15 | Jiang et al., CANCER RES. (1996) 56: 3534-3539 |
| D3S1581 | 3p21.2-14.2 | Jones et al., ONCOGENE (1992) 7: 1631-1634 |
| D6S448 | 6q25.1-25.2 | Colitti et al., ONCOGENE (1998) 16: 555-559 |
| D12S354 | 12q23ter | Hatta et al., BR. J. CANCER (1997) 75: 1256-1262 |
| D5S424 | 5q13.1-21 | Tavassoli et al., BR. J. CANCER (1996) 74: 115-119 |
| BRCA2 | 13q12 | Foster et al., CANCER RES. (1996) 56: 3622-3625 |
| APC | 5q21-22 | Tavassoli et al., BR. J. CANCER (1996) 74: 115-119 |
| p53 | 17p13.3 | Wertheim et cd., ONCOGENE (1996) 12: 2147-2153 |
| D17S579 | 17q21(BRCA1) | Wertheim et al., ONCOGENE (1996) 12: 2147-2153 |
| D22S284 | 22q12 | Bryan et al., CANCER RES. (1996) 56: 719-721 |
| DCC | 18q21 | Cliby et al., CANCER RES. (1993) 53: 2393-2398 |
| D7S1805 | 7q35 | Nakayama et al., INT. J. CANCER (2001) 94: 605-609 |
| D3S2403 | 3p25.1-25.2 | Nakayama et al., INT. J. CANCER (2001) 94: 605-609 |
| D9S922 | 9q21.32 | Nakayama et al., INT. J. CANCER (2001) 94: 605-609 |

TABLE B

| | Chromosomal | |
|---|---|---|
| Locus | Location | Reference |
| D7S480, D7S523 | 7q31 | Fromont et al., J. UROL. (2003) 170: 1394-1397; |
| | | Valeri et al., UROL. ONCOL. (2005) 23: 87-92 |
| D8S261, D8S286 | 8p22 | Fromont et al., J. UROL. (2003) 170: 1394-1397; |
| | | Valeri et al., UROL. ONCOL. (2005) 23: 87-92 |
| D12S89, D12S98 | 12p13 | Fromont et al., J. UROL. (2003) 170: 1394-1397; |
| | | Valeri et al., UROL. ONCOL. (2005) 23: 87-92 |
| D13S153, D13S273 | 13q14 | Fromont et al., J. UROL. (2003) 170: 1394-1397; |
| | | Valeri et al., UROL. ONCOL. (2005) 23: 87-92 |
| D16S518, | 16q23.2 | Fromont et al., J. UROL. (2003) 170: 1394-1397; |
| D16S3097 | | Valeri et al., UROL. ONCOL. (2005) 23: 87-92 |
| D18S39, D18S1144 | 18q21 | Fromont et al., J. UROL. (2003) 170: 1394-1397; |
| | | Valeri et al., UROL. ONCOL. (2005) 23: 87-92 |

Thus in some embodiments the invention provides a microarray with both random markers and hotspot markers (i.e., probes directed to specific markers where LOH is known to be associated with a particular clinical outcome). In some embodiments the array probes are more densely packed around the hotspot markers. Thus the resolution of the array within areas of special interest may be increased to beyond 4 Kb, 3 Kb, 2 Kb or 1 Kb or greater by using probes directed staggered more tightly across these regions (while optionally omitting probes to areas of lesser interest). In some embodiments the invention provides a method comprising determining the amount of LOH in a patient sample and determining whether the sample has LOH in at least one of the genes or genomic regions listed in Table 8. In some embodiments the invention provides a method of classifying cancer comprising determining the amount of LOH in a patient sample and determining whether the sample has LOH in at least one of the genes or genomic regions listed in Table 8, wherein either an increased amount of LOH or LOH in any of the genes or genomic regions listed in Table 8 indicates a poor prognosis.

In some embodiments the genome-wide SNP analysis comprises analyzing at least 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, 900,000, 1,000,000, 1,250,000, 1,500,000, 1,750,000, 2,000,000, or more SNPs (see Examples 1 & 4). In preferred embodiments, the genome-wide SNP analysis comprises analyzing more than 5,000, more preferably more than 10,000 SNPs. In some embodiments the resolution of the platform used to analyze SNP markers (e.g., microarray) is 1,000,000, 500,000, 250,000, 100,000, 50,000, 25,000, 10,000, 9,000, 8,000, 7,000, 6,000, 5,000, 4,000, 3,000, 2,000, 1,000, 900, 800, 700, 600, 500, 400, 300, 250, 200, 250, 200, 150, 100, 75, 50, 40, 30, 20, 10, 6, or 5 Kb or less. As used in the context of LOH analysis, “resolution” refers to the smallest chromosomal region of copy number variation (e.g., deletion or amplification) that may be detected, on average, by a particular platform or technique. In SNP microarrays, for instance, resolution is expressed as the average distance along a chromosome or genome between two SNP markers. Resolution in whole-genome SNP microarrays can be as low as 5 Kb or less. This generally means that along the entire genome, within an average stretch of 5 Kb or more, the microarray has probes directed to at least two different loci (e.g., SNPs). In some embodiments each of the different probes on the array is an oligonucleotide from 15 to 200, 15 to 150, 15 to 100, 15 to 75, 15 to 60, or 20 to 55 bases in length.

Various techniques may be used to determine the amount of LOH in a sample. In some embodiments determining the amount of LOH in a sample comprises isolating nucleic acid from the sample and analyzing the nucleic acid to determine the amount of LOH. One technique for detecting LOH is array-based comparative genomic hybridization (a-CGH), described in U.S. Pat. Nos. 5,830,645 and 6,562,565. a-CGH involves competitive hybridization between labeled test DNA or normal reference DNA and nucleic acid probes arrayed on a solid support. Chromosomal regions in the test DNA at increased or decreased copy number as compared to the normal reference DNA are identified by detecting regions where the ratio of signal from the two different colors is altered.

LOH can also be determined using microarrays. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes,” which function to hybridize or bind with a sample material (e.g., nucleic acids prepared or obtained from the tumor samples), in hybridization experiments. The reverse situation can also be applied: the sample can be bound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude of different oligonucleotide probes (e.g., hundreds, thousands, hundreds of thousands or even millions of probes scattered across the genome) to analyze LOH at a multitude of loci at once. SNP microarrays, for example, allow for high density, whole-genome analysis. The number of SNP loci at which LOH is found can give, according to the present invention, an indication of the aggregate genomic amount of LOH. This in turn can classify the cancer from which the sample was derived.

As a preferred embodiment, microarray LOH analysis provides excellent resolution. SNP microarrays for example can give resolutions as high as 6 Kb. See, e.g., Product Page for Genome-Wide Human SNP Array 6.0® chip by Affymetrix® (available at Affymetrix® website). Such high resolution is important when one considers that many incidents of LOH may not involve much more than a few Kb of the genomic DNA. Numerous examples of such small mutations may indicate dangerous genomic instability but may go undetected using traditional LOH analysis.

General principles for the design, construction and use of microarrays are well-known in the art and are discussed in the following sources: Guo et al., NUCLEIC ACIDS RES. (1994) 22:5456-65; Maskos & Southern, NUCLEIC ACIDS RES. (1992) 20:1679-84; Southern et al., NUCLEIC ACIDS RES. (1994) 22:1368-73; U.S. Pat. Nos. 5,137,765, 5,143,854, 5,242,974, 5,252,743, 5,266,222, 5,324,633, 5,384,261, 5,405,783, 5,412,087, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,889,165, 5,936,324, 5,959,098, 5,968,740, 5,974,164, 5,981,185, 5,981,956, 6,025,601, 6,033,860, 6,040,193, 6,090,555, 6,136,269, 6,147,205, 6,262,216, 6,269,846, 6,310,189, 6,428,752, and 6,649,348; PCT Application/International Publication Nos. WO/1999/036760, WO/2000/058516, and WO/2001/058593; all of which are incorporated herein by reference in their entirety. Nucleic acid arrays useful in the present invention include, but are not limited to, those that are commercially available from Affymetrix (Santa Clara, Calif.), e.g., Affymetrix 500K SNP arrays. Example arrays are shown on the website at affymetrix.com. Another microarray supplier is illumina of San Diego, Calif. with example arrays shown on their website at illumina.com.

Array-based LOH analysis according to the present invention will often require hybridization, e.g., hybridization of a nucleic acid probe to a nucleic acid target, under specified conditions. Methods for conducting polynucleotide hybridization assays are well developed in the art. Hybridization assay procedures and conditions used in the methods of the invention will vary depending on the application and are selected in accordance with the general binding methods known including those referred to in: Maniatis et al., Molecular Cloning: A Laboratory Manual (2d Ed. Cold Spring Harbor, N.Y., 1989); Berger & Kimmel, Methods in Enzymology, Vol. 152, Guide to Molecular Cloning Techniques (Academic Press, Inc., San Diego, Calif., 1987); Young & Davis, PROC. NAT. ACAD. SCI. (1983) 80:1194. Methods and apparatus for carrying out repeated and controlled hybridization reactions have been described in U.S. Pat. Nos. 5,871,928, 5,874,219, 6,045,996 and 6,386,749, 6,391,623, each of which is incorporated herein by reference.

A related method of detecting LOH is quantitative PCR™ (gPCR™) gPCR™ is described in detail by Freeman et al., BIOTECHNIQUES (1999) 26:112-125. In this method, one uses primers to amplify regions of interest and either calculates the relative amounts of amplified product afterwards or tracks these relative amounts in real-time during the reaction. Either way, because PCR™ amplifies nucleic acids in a template-dependent manner, a relative difference in the amount of amplified product directly correlates to a relative difference in the initial number of nucleic acid templates. Thus, by detecting a difference in the initial number of templates, gPCR™ is in essence a form of LOH analysis and is one technique that may be used in the practice of the present invention to determine the amount of LOH.

The term “high stringency hybridization conditions,” when used in connection with nucleic acid hybridization, means those conditions in a hybridization reaction known in the art to allow only for specific hybridization between nucleic acids of the same or highly similar sequence and to not allow for non-specific hybridization. For example, high stringency conditions are generally required in a SNP array because there is usually only one base-pair difference between the test nucleic acid and its target probe. An example includes hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. Lower stringency conditions may be used in different circumstances—i.e., when not dealing with a SNP—as understood by one skilled in the art.

The methods of the invention may also involve signal detection of hybridization between ligands after (and/or during) hybridization. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625; U.S. Ser. No. 10/389,194; and PCT Application PCT/US99/06097 (published as WO/1999/047964); each of which is hereby incorporated by reference in its entirety. Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639, 6,218,803, and 6,225,625; U.S. Ser. Nos. 10/389,194, 60/493,495; and PCT Application PCT/US99/06097 (published as WO/1999/047964); each of which is hereby incorporated by reference in its entirety.

For the purpose of comparing two different nucleic acid or polypeptide sequences, one sequence (test sequence) may be described to be a specific “percentage identical to” another sequence (comparison sequence) in the present disclosure. In this respect, the percentage identity may be determined by the algorithm of Karlin and Altschul, Proc. Natl. Acad. Sci. USA, 90:5873-5877 (1993), which is incorporated into various BLAST programs. Specifically, the percentage identity may be determined by the “BLAST 2 Sequences” tool, which is available at NCBI's website. See Tatusova and Madden, FEMS Microbiol. Lett., 174(2):247-250 (1999). For pairwise DNA-DNA comparison, the BLASTN 2.1.2 program may be used with default parameters (Match: 1; Mismatch: -2; Open gap: 5 penalties; extension gap: 2 penalties; gap x_dropoff: 50; expect: 10; and word size: 11, with filter). For pairwise protein-protein sequence comparison, the BLASTP 2.1.2 program may be employed using default parameters (Matrix: BLOSUM62; gap open: 11; gap extension: 1; x_dropoff: 15; expect: 10.0; and wordsize: 3, with filter).

This is particularly important in the embodiments of the present invention where whole genome sequencing is used to determine the amount of LOH. In such embodiments, the entire genomic sequence or some sizable portion thereof from a patient sample may be compared to a consensus or other reference sequence and a percentage identity between the two sequences may be determined. If the percent identity drops below a certain threshold index level, i.e., if the level of LOH rises above some threshold amount, then it may be concluded that the patient has, e.g., an increased likelihood of shorter survival, poor prognosis, etc.

In some embodiments the patient has a particular cancer for which LOH is predictive. Example 1 demonstrates using LOH amount for determining prognosis in ovarian cancer while Example 3 shows using LOH amount for determining prognosis in breast cancer. In some embodiments the invention provides a method of determining a cancer patient's prognosis comprising determining the amount of LOH in a sample containing cancer cells from said patient, wherein the patient has a cancer chosen from the group consisting of ovarian, breast, lung, prostate and colon, and wherein high LOH indicates a poor prognosis. In some embodiments the cancer is ovarian cancer. In some embodiments the cancer is breast cancer. In some embodiments the cancer is lung cancer. In some embodiments the cancer is prostate cancer. In some embodiments the cancer is colon cancer. In some embodiments the cancer is ovarian cancer and the loci analyzed do not include those listed in Table A. In some embodiments the cancer is prostate cancer and the loci analyzed do not include those listed in Table B.

When measuring overall genomic stability, the particular loci assayed for LOH are often not as important as the total number of loci. The number of loci assayed can vary depending on many factors, e.g., technical constraints, cost considerations, the classification being made, the cancer being tested, the desired level of predictive power, etc. Increasing the number of loci assayed according to the invention is, as a general matter, advantageous because, inter alia, a larger pool of loci to be assayed means less “noise” caused by outliers and less chance of an assay error throwing off the overall predictive power of the test. However, cost and other considerations will sometimes limit this number (especially in the case of microsatellite markers) and finding the optimal number of loci for LOH analysis is desirable.

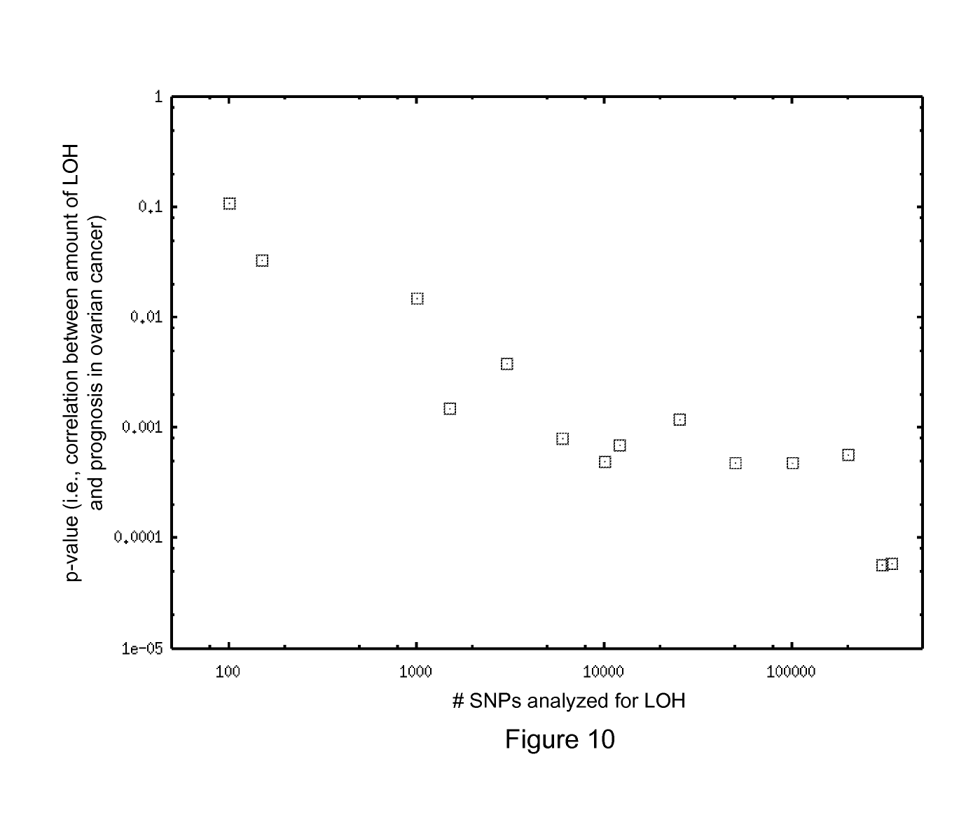

It has been discovered that the predictive power of an overall LOH level assay often ceases to increase significantly beyond a certain number of loci (see FIG. 10; Example 4). Example 4 and FIG. 10 illustrate the empirical determination of optimal numbers of loci in methods of the invention. Randomly selected subsets of the SNPs contained on the Affymetrix 500K GeneChip™ microarray were tested as distinct SNP “panels” and predictive power (i.e., p-value) was determined for each. As FIG. 10 shows, p-values gained significance at about 150 to 200 loci and ceased to improve significantly between about 5,000 and about 15,000 loci, thus indicating that a minimum number of loci in a prognostic assay is from about 150 to about 200 and a preferred number of loci is between about 5,000 and about 15,000. Thus some embodiments of the invention provide a method of classifying cancer (e.g., predicting prognosis) in a patient (e.g., breast or ovarian cancer patient) comprising determining LOH in at least 150 loci, wherein an increased amount of overall LOH across these loci indicates a poor prognosis. In some embodiments the amount of LOH is determined by analyzing at least 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, 900,000, 1,000,000, 1,250,000, 1,500,000, 1,750,000, 2,000,000, or more loci.

In some embodiments the amount of LOH is determined by analyzing between 150 and 15,000, 200 and 15,000, 250 and 15,000, 300 and 15,000, 350 and 15,000, 400 and 15,000, 450 and 15,000, 500 and 15,000, 550 and 15,000, 600 and 15,000, 650 and 15,000, 700 and 15,000, 750 and 15,000, 800 and 15,000, 850 and 15,000, 900 and 15,000, 950 and 15,000, 1,000 and 15,000, 1,250 and 15,000, 1,500 and 15,000, 1,750 and 15,000, 2,000 and 15,000, 2,500 and 15,000, 3,000 and 15,000, 3,500 and 15,000, 4,000 and 15,000, 4,500 and 15,000, 5,000 and 15,000, 6,000 and 15,000, 7,000 and 15,000, 8,000 and 15,000, 9,000 and 15,000, 10,000 and 15,000, 1,000 and 150,000, 1,250 and 150,000, 1,500 and 150,000, 1,750 and 150,000, 2,000 and 150,000, 2,500 and 150,000, 3,000 and 150,000, 3,500 and 150,000, 4,000 and 150,000, 4,500 and 150,000, 5,000 and 150,000, 6,000 and 150,000, 7,000 and 150,000, 8,000 and 150,000, 9,000 and 150,000, 10,000 and 150,000, 15,000 and 150,000, 20,000 and 150,000, 25,000 and 150,000, 30,000 and 150,000, 35,000 and 150,000, 40,000 and 150,000, 45,000 and 150,000, 50,000 and 150,000, 60,000 and 150,000, 70,000 and 150,000, 80,000 and 150,000, 90,000 and 150,000, or 100,000 and 150,000 loci. In some embodiments the method further comprises analyzing at least one additional marker that significantly increases the predictive power of the panel.

Algorithms may be used to determine an optimal number of loci to be assayed in determining the amount of LOH in a sample. More specifically, the optimal number of loci (n_{O}) can be found wherever the following is true

(Pₙ₊₁−Pₙ)<C_{O},

wherein P is the predictive power (i.e., Pₙ is the predictive power of an assay with n loci and Pₙ₊₁ is the predictive power of an assay with n+1 loci) and C_{O} is some optimization constant. Predictive power can be defined in many ways known to those skilled in the art including, but not limited to, the signature's p-value. C_{O} can be chosen by the artisan based on his or her specific constraints. For example, if cost is not a critical factor and extremely high levels of sensitivity and specificity are desired, C_{O} can be set very low such that only trivial increases in predictive power are disregarded. On the other hand, if cost is decisive and moderate levels of sensitivity and specificity are acceptable, C_{O} can be set higher such that only significant increases in predictive power warrant increasing the number of genes in the signature. In some embodiments n is such that (Pₙ₊₁−Pₙ) is less than 0.01, 0.009, 0.008, 0.007, 0.006, 0.005, 0.004, 0.003, 0.002, 0.001, 0.0005, 0.0001, 0.00005, 0.00001, or less.

Sometimes it is helpful to express the change in predictive power as a fraction of the initial predictive power, as follows:

(Pₙ₊₁−Pₙ)/Pₙ<C_{O′},

wherein C_{O′} is a new optimization constant usually expressed as a percentage. In some embodiments n is such that (Pₙ₊₁−Pₙ)/Pₙ is less than 50%, 45%, 40%, 35%, 30%, 25%, 20%, 15%, 10%, 9%, 8%, 7%, 6%, 5%, 4%, 3%, 2%, 1%, 0.1%, 0.01%, 0.001%, or less.

Alternatively, a graph of predictive power as a function of LOH loci number may be plotted (as in FIG. 10) and the second derivative of this plot taken. The point at which the second derivative decreases to some predetermined value (C_{O″}) may be the optimal number of genes in the signature.

Another aspect of the invention provides systems and computer-implemented methods of classifying cancer. Thus the invention provides a system for determining a cancer patient's prognosis, comprising:
(1) a sample analyzer for determining the level of overall LOH in a cancer sample, wherein the sample analyzer contains the cancer sample, DNA from the cancer sample, or DNA synthesized from the DNA from the cancer sample;
(2) a first computer program for receiving overall LOH level data as determined by the sample analyzer; and
(3) a second computer program for comparing the overall LOH level data to one or more reference values each associated with a predetermined prognosis.
   In some embodiments the system comprises a computer program for determining the patient's prognosis and/or determining (including quantifying) the patient's degree of risk of cancer recurrence or progression based at least in part on the comparison of the test value with said one or more reference values. In some embodiments one computer program performs all of the above functions (e.g., receiving LOH data, comparing LOH data to reference/index value, and determining patient's prognosis). In some embodiments the sample analyzer contains reagents for determining overall LOH levels in the sample. In some embodiments the sample analyzer contains SNP-specific reagents as described above.

In some embodiments, the system further comprises a display module displaying the comparison between the overall LOH level in the sample and the one or more reference values, or displaying a result of the comparing step, or displaying the patient's prognosis and/or degree of risk of cancer recurrence or progression.

The sample analyzer can be any instrument useful in determining copy number and/or LOH, including, e.g., a sequencing machine (e.g., Illumina HiSeg™, Ion Torrent PGM, ABI SOLiD™ sequencer, PacBio RS, Helicos Heliscope™, etc.), a real-time PCR machine (e.g., ABI 7900, Fluidigm BioMark™, ABI OpenArray system, Wafergen SmartChip system, etc.), a microarray instrument (e.g., Affymetrix GeneChip3000 or GeneTitan analyzers, Illumina iScan or Bead Express analyzers), etc. A sample analyzer could even include the Luminex xMAP system.

The invention further provides a computer-implemented method of classifying cancer in a patient comprising:
accessing information on said patient contained in a computer-readable medium;
querying the data stored in said computer-readable medium to obtain LOH information for a sample from said patient;
determining whether said LOH information indicates said sample has high LOH; and
outputting or displaying that said patient has a poor prognosis if said sample has high LOH.

Techniques for analyzing copy number and LOH will often be implemented using hardware, software or a combination thereof in one or more computer systems or other processing systems capable of effectuating such analysis. Thus one aspect of the present invention provides a system for determining a cancer patient's prognosis. Generally speaking, the system comprises (1) computer program for receiving, storing, and/or retrieving a patient's LOH data; (2) computer program for querying this patient data; (3) computer program for concluding whether the patient has a poor prognosis based on this patient data; and (4) computer means (e.g., output module) for outputting/displaying this conclusion. In some embodiments this means for outputting the conclusion may comprise a computer means for informing a health care professional of the conclusion.

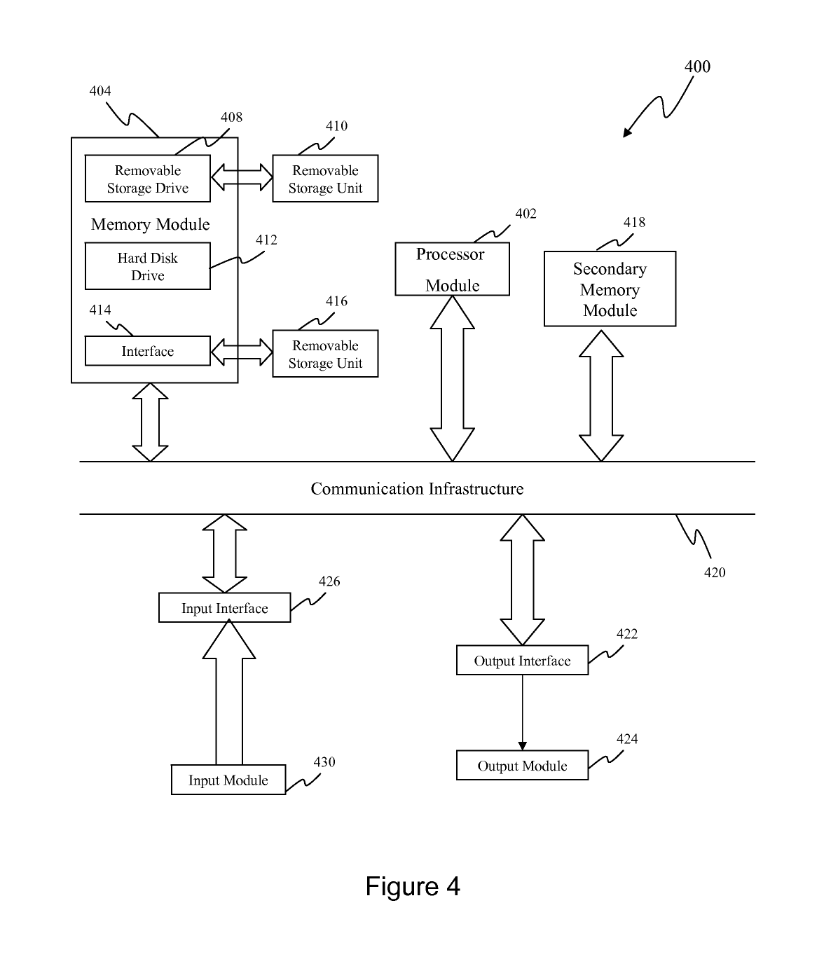

One example of such a system is the computer system [400] illustrated in FIG. 4. Computer system [400] may include at least one input module [430] for entering patient data into the computer system [400]. The computer system [400] may include at least one output module [424] for indicating whether a patient has an increased or decreased likelihood of response and/or indicating suggested treatments determined by the computer system [400]. Computer system [400] may include at least one memory module [406] in communication with the at least one input module [430] and the at least one output module [424].

The at least one memory module [406] may include, e.g., a removable storage drive [408], which can be in various forms, including but not limited to, a magnetic tape drive, a floppy disk drive, a VCD drive, a DVD drive, an optical disk drive, etc. The removable storage drive [408] may be compatible with a removable storage unit [410] such that it can read from and/or write to the removable storage unit [410]. Removable storage unit [410] may include a computer usable storage medium having stored therein computer-readable program codes or instructions and/or computer readable data. For example, removable storage unit [410] may store patient data. Example of removable storage unit [410] are well known in the art, including, but not limited to, floppy disks, magnetic tapes, optical disks, and the like. The at least one memory module [406] may also include a hard disk drive [412], which can be used to store computer readable program codes or instructions, and/or computer readable data.

In addition, as shown in FIG. 4, the at least one memory module [406] may further include an interface [414] and a removable storage unit [416] that is compatible with interface [414] such that software, computer readable codes or instructions can be transferred from the removable storage unit [416] into computer system [400]. Examples of interface [414] and removable storage unit [416] pairs include, e.g., removable memory chips (e.g., EPROMs or PROMs) and sockets associated therewith, program cartridges and cartridge interface, and the like. Computer system [400] may also include a secondary memory module [418], such as random access memory (RAM).

Computer system [400] may include at least one processor module [402]. It should be understood that the at least one processor module [402] may consist of any number of devices. The at least one processor module [402] may include a data processing device, such as a microprocessor or microcontroller or a central processing unit. The at least one processor module [402] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [402] may include any other type of analog or digital circuitry that is designed to perform the processing functions described herein.

As shown in FIG. 4, in computer system [400], the at least one memory module [406], the at least one processor module [402], and secondary memory module [418] are all operably linked together through communication infrastructure [420], which may be a communications bus, system board, cross-bar, etc.). Through the communication infrastructure [420] computer program codes or instructions or computer readable data can be transferred and exchanged. Input interface [426] may operably connect the at least one input module [426] to the communication infrastructure [420]. Likewise, output interface [422] may operably connect the at least one output module [424] to the communication infrastructure [420].

The at least one input module [430] may include, for example, a keyboard, mouse, touch screen, scanner, and other input devices known in the art. The at least one output module [424] may include, for example, a display screen, such as a computer monitor, TV monitor, or the touch screen of the at least one input module [430]; a printer; and audio speakers. Computer system [400] may also include, modems, communication ports, network cards such as Ethernet cards, and newly developed devices for accessing intranets or the internet.

The at least one memory module [406] may be configured for storing patient data entered via the at least one input module [430] and processed via the at least one processor module [402]. Patient data relevant to the present invention may include copy number or LOH information. Patient data relevant to the present invention may also include clinical parameters relevant to the patient's disease. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [406] may include a computer-implemented method stored therein. The at least one processor module [402] may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient as having or not having a poor prognosis for survival. For example, the computer-implemented method may be configured to inform a physician that a particular patient has a poor prognosis for survival. Alternatively or additionally, the computer-implemented method may be configured to actually suggest a particular course of treatment based on the answers to/results for various queries.

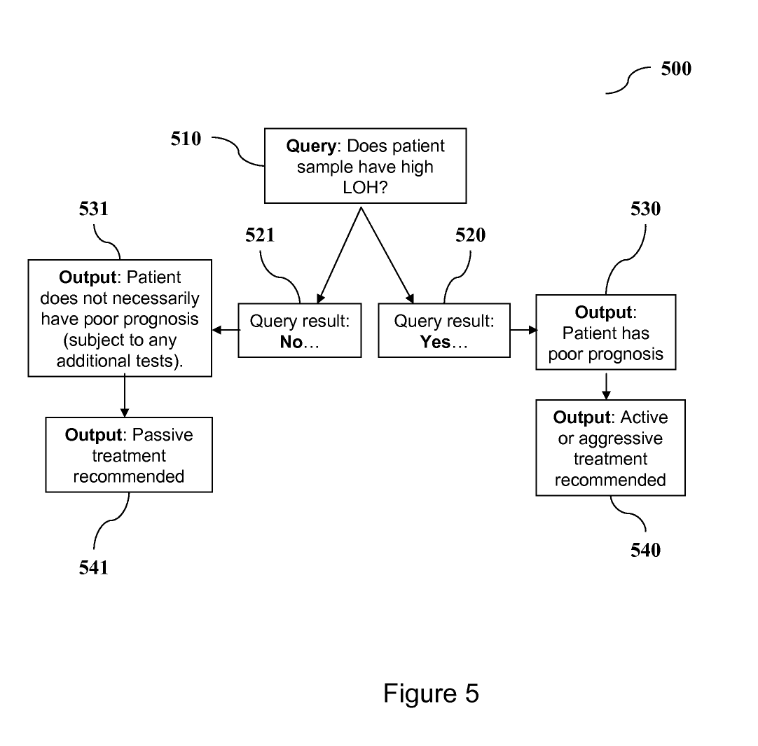

FIG. 5 illustrates one embodiment of a computer-implemented method [500] of the invention that may be implemented, e.g., with the computer system (see FIG. 4) of the invention. The method [500] begins with a query [510] whether the amount of overall LOH in a sample obtained from the patient is high. If the answer to/result for this query is “Yes” [520], the method concludes [530] that the patient has a poor prognosis. If the answer to/result for this query is “No” [521], the method concludes [531] that the patient does not necessarily have a poor prognosis (subject to any additional tests/results the health care provider may want to run/review). The method [500] may then proceed with more queries, make a particular treatment recommendation ([540], [541]), or simply end.

In some embodiments, the computer-implemented method of the invention [500] is open-ended. In other words, the apparent first step [510] in FIG. 5 may actually form part of a larger process and, within this larger process, need not be the first step/query. Additional steps may also be added onto the core methods discussed above. These additional steps include, but are not limited to, informing a health care professional (or the patient itself) of the conclusion reached; combining the conclusion reached by the illustrated method [500] with other facts or conclusions to reach some additional or refined conclusion regarding the patient's diagnosis, prognosis, treatment, etc.; making a recommendation for treatment (e.g., “patient should/should not undergo aggressive chemotherapy”); additional queries about additional biomarkers, clinical parameters, or other useful patient information (e.g., age at diagnosis, general patient health, etc.).

Regarding the above computer-implemented method [500], the answers to the queries may be determined by the method instituting a search of patient data for the answer. For example, to answer the query [510], patient data may be searched for LOH data. If such a comparison has not already been performed, the method may compare these data to some reference in order to determine if the patient has an increased amount of LOH. Additionally or alternatively, the method may present one or more of the queries [510] to a user (e.g., a physician) of the computer system ([400] in FIG. 4). For example, the question [510] may be presented via an output module [424]. The user may then answer “Yes” or “No” via an input module [430]. The method may then proceed based upon the answer received. Likewise, the conclusions [530, 531] may be presented to a user of the computer-implemented method via an output module [424].

Thus in some embodiments the invention provides a method comprising: accessing information on a patient sample's level of overall LOH stored in a computer-readable medium; querying this information to determine whether the sample has high overall LOH; and outputting [or displaying] the sample's LOH status (e.g., high). As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a sample's/patient's overall LOH status.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present invention may have embodiments that include methods for providing genetic information over networks such as the Internet as shown in U.S. Ser. Nos. 10/197,621 (U.S. Pub. No. 20030097222); 10/063,559 (U.S. Pub. No. 20020183936), 10/065,856 (U.S. Pub. No. 20030100995); 10/065,868 (U.S. Pub. No. 20030120432); 10/423,403 (U.S. Pub. No. 20040049354).

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an LOH assay is conducted outside the United States, the information and data on a test result (i.e., amount of LOH) may be generated, cast in a transmittable form as described above, and then imported into the United States. Accordingly, the present invention also encompasses a method for producing a transmittable form of information on level of LOH for at least one patient sample. The method comprises the steps of (1) determining the level of overall LOH in a patient sample according to methods of the present invention; and (2) embodying the result of the determining step in a transmittable form. The transmittable form is the product of such a method.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out LOH analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Yet another aspect of the invention provides methods of treatment utilizing information on the amount of LOH. Thus the invention provides a method of treatment comprising determining the amount of LOH in a sample containing cancer cells from said patient and administering, prescribing or recommending an aggressive treatment if said sample has high LOH. Whether a treatment is aggressive or not will generally depend on the cancer-type, the age of the patient, etc. For example, in breast cancer adjuvant chemotherapy is a common aggressive treatment given to complement the less aggressive standards of surgery and hormonal therapy. Those skilled in the art are familiar with various other aggressive and less aggressive treatments for each type of cancer.

In some embodiments, the inventive methods provide for specific sample preparation. Depending on the platform (e.g., microarrays generally or a specific microarray) and the experiment to be performed, sample nucleic acid can be prepared in a number of ways by methods known to the skilled artisan. In some embodiments, prior to or concurrent with analysis of copy number profiles nucleic acids in a sample may be amplified by any number of mechanisms. The most common amplification procedure used involves PCR™. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., NUCLEIC ACIDS RES. (1991) 19:4967; Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675; each of which is incorporated herein by reference in their entireties. In some embodiments, the sample may be amplified on the array (e.g., U.S. Pat. No. 6,300,070 which is incorporated herein by reference).

Additional methods of sample preparation and techniques for reducing the complexity of a nucleic sample are described in Dong et al., GENOME RES. (2001) 11:1418, in U.S. Pat. Nos. 6,361,947, 6,391,592 and U.S. patent application Ser. Nos. 09/916,135, 09/920,491, 09/910,292, 10/013,598, U.S. Patent Application Publication Nos. 2003/0096235 & 2003/0082543.

It will often be desirable to minimize the amount of non-tumor cell contamination in the patient's sample. In some embodiments the sample is non-tumor cell contamination is below some desired level (e.g., to below 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less). Various techniques for doing this are known in the art. For example, in some embodiments the level of contamination is reduced to the desired level using microdissection.

Although a high level of contamination with normal DNA makes LOH analysis challenging, at some level the contamination can be helpful for the analysis when a normal paired sample is not available. In this case even one SNP that is homozygous in cancer cells and heterozygous in normal cells can in theory signify a region with LOH. In practice, a few such SNPs can reliably identify relatively small regions of LOH. In contrast, in the absence of any contamination with normal cells, such as the case with cancer cell lines, one may need a long stretch of homozygous SNPs to reliably identify LOH regions. Thus in some embodiments the amount of non-tumor cell contamination in the sample is between 1% and 10%, 2% and 10%, 3% and 10%, 4% and 10%, 5% and 10%, 6% and 10%, 7% and 10%, 8% and 10%, 9% and 10%, or 10% and 15%. In some of these embodiments the method comprises analyzing only tumor samples from the patient (i.e., no paired normal sample). In some embodiments the amount of non-tumor cell contamination in the sample is less than 55%.

Another aspect of the invention provides methods (including computer-implemented methods) and systems for accurately determining the copy number of a locus (including LOH) for cancer cells within a sample having significant normal cell contamination. Several algorithms have been developed to detect LOH regions using information from SNP arrays. See, e.g., Goransson et al., PLOS ONE (2009) 4:e6057; Huang et al., BMC BIOINFORMATICS (2006) 7:83; Ishikawa et al., BIOCHEM. BIOPHYS. RES. COMMUN. (2005) 333:1309-1314; Laframboise et al., BIOSTATISTICS (2007) 8:323-836; Laframboise et al., PLOS COMPUT. BIOL. (2005) 1:e65; Nannya et al., CANCER RES. (2005) 65:6071-6079; Zhao et al., CANCER RES. (2004) 64:3060-3071. These algorithms often fail to explicitly take into account contamination of tumor samples with benign tissue. This contamination often is large enough to make the detection of LOH regions challenging. For example, if the observed ratio of the signals of two alleles, A and B, is 2:1, there are two possibilities. On one hand, the cancer cells may have LOH with deletion of allele B in a sample with 50% contamination with normal cells. On the other hand, it is possible there is no LOH and instead allele A is duplicated in a sample with no contamination with normal cells. While Goransson et al. teach a method to take into account the normal tissue contamination, this method is limited, inter alia, to chromosomal regions with two copies. In reality, as the Examples below show, LOH often happens in chromosomal regions with three or more copies.

The inventors have developed a novel computational method to detect LOH regions in cancer cell lines and tumors. The method assumes a prior knowledge of copy number regions as well as the level of contamination with the normal DNA. This information can be obtained using the techniques described in Example 2. This approach enables reliable detection of LOH within regions with copy number greater than two. In some embodiments, because of the significant contamination with benign tissue, LOH detection in tumor samples is limited to loci with copy number more than 2 but less than 10, less than 9, less than 8, less than 7, less than 6, less than 5, less than 4, or less than 3 (with fractional copy number possible). This aspect of the invention is particularly useful since our results show that LOH is much more frequently observed within amplified regions (e.g., copy number greater than two) than was assumed previously (Goransson et al.). In other words, regions of amplification were often either excluded from LOH analysis or copy number was imprecisely measured such that regions of amplification made LOH analysis inaccurate.

Thus in some embodiments the invention provides a method of determining the amount of LOH in a sample comprising determining the copy number for at least 100 loci, wherein those loci having copy number greater than two are factored into said amount of LOH. In some embodiments copy number is determined for at least 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, 900,000, 1,000,000, 1,250,000, 1,500,000, 1,750,000, 2,000,000, or more loci. In some embodiments at least 1%, 2%, 3%, 4%, 5%, 6%, 7%, 8%, 9%, 10%, 15%, 20%, or 25% or more of the loci for which copy number is determined show copy number greater than two. In some embodiments copy number is determined according to the techniques described in Example 2.

### Example 1

This Example presents a computational method to detect LOH regions in cancer cell lines and tumors using Affymetrix 500K GeneChip oligonucleotide arrays. The method assumes a prior knowledge of copy number regions as well as the level of contamination with the normal DNA, which can be determined according to the methods discussed below in Example 2 or according to any technique known in the art. Hidden Markov Model was used to obtain the most likely LOH regions.

Our approach enables reliable detection of LOH within regions with copy number below five in cancer cell lines. Because of the significant contamination with benign tissue, LOH detection in tumor samples is limited to chromosomal regions with copy number below four. Our results show that LOH is much more frequently observed within amplified regions than was assumed previously (Goransson et al.).

The results of copy number (CN) analysis for 178 cancer cell lines (including 44 breast cancer cell lines, 35 colon cancer cell lines, and 19 brain cancer cell lines) as well as for 100 ovarian tumors are presented below. We found that in ovarian tumors the fraction of genome with LOH (FGLOH) is strongly correlated with tumor grade and survival. Thus FGLOH may be used as a prognostic and/or predictive marker for ovarian cancer.

### Materials and Methods

Genomic DNA

Frozen tumor was cut into 10 μm sections and macrodissected to minimize contamination with normal tissue. A QIAamp DNA Mini Kit™ (QIAgen) was used to isolate the DNA as per the manufacturer's protocol with an overnight lysis incubation at 56° C. and including the optional RNase A treatment.

Affymetrix 500K GeneChip™

The Affymetrix GeneChip Mapping™ NspI or StyI Assay Kit was used in the generation of biotinylated DNA for Affymetrix Mapping 500K™ NspI or StyI microarray hybridizations (each assay was prepared separately). Genomic DNA (250 ng) was digested with NspI or StyI restriction enzyme and adaptors were added to restriction fragment ends with T4 DNA ligase. Adaptor-modified samples were PCR™ amplified using Clontech Titanium Taq™, which generated an amplified product of average size between 200 and 1,100 bp. Amplification products were purified using a Clontech DNA amplification cleanup kit. 90 μg of purified DNA was fragmented using Affymetrix Fragmentation Reagent™. Biotin-labeling of the fragmented sample was accomplished using the GeneChip DNA Labeling Reagent™. Biotin-labeled DNA was hybridized on NspI or StyI Affymetrix microarrays at 49° C. for 16 to 18 hours in the Affymetrix rotation oven. After hybridization, probe array wash and stain procedures were carried out on the automatic Affymetrix Fluidics Stations as per manufacturer's manual and microarrays were scanned and raw data was collected by Affymetrix GeneChip Scanner 3000™.

Combining Signals from Multiple Probes on Affymetrix 500K GeneChip™ Arrays

Affymetrix 500K GeneChip™ array contains 25-mer oligonucleotides distributed over two subarrays, Nsp1 and Sty1 containing 262,264 and 238,304 SNPs, respectively. For each SNP Affymetrix software provides users with signal intensities for either six or ten oligonucleotide probe quartets consisting with perfect-match (PM) and mismatch (MM) pairs for both alleles. The first task is to accurately estimate the dosage of each allele by optimally combining the signals from individual probes. In order to accomplish that we used genotyping data for 48 cell lines provided by Affymetrix. Because these cell lines were collected from the normal, non-cancerous, tissue we assume that for essentially all SNPs copy number will be equal to two with the exception of X chromosome for males.

We assumed that signal intensity of mismatched probes is equal to the noise level of corresponding perfectly matched probes. Thus for each quartet and allele, the intensity I_{MM} from the MM probe is subtracted from the intensity I_{PM} from the PM probe: ΔI=I_{PM}−I_{MM}. The next step is to use signals from both alleles, A and B, within a quartet q to calculate an angle θ_{q} in the rectangular triangle with sides equal to ΔI_{A} and ΔI_{B}:

θ_{q}=arc tan(ΔI_{B}/ΔI_{A})

The angle is expected to be close to zero if only allele A is present and close to π/2 if only allele B is present. Then we calculated median value of these angles for each genotype in the collection of 48 cell lines: θ_{qAA}, θ_{qAB}, and θ_{qBB}. The median values were used to estimate how accurately the dosage of each allele can be determined using data from each quartet:

σ_{q}²=(1/N)Σ_{S}(θ_{q}−θ_{qG})²/θ_{qW}−θ_{qG})²

Here the sum is over all N=48 cell lines, G is the genotype of a cell line (G=AA, AB, or BB), W is the alternative genotype defined as

W=AB if G=AA or BB

W=AA if G=AB and θ_{q}<θ_{qAB}

W=BB if G=AB and θ_{q}>θ_{qAB}

The values θ_{q} for all quartets for SNP i are linearly combined to determine allele dosage with weights reflecting performance of individual quartets: θᵢ=Σ_{q}w_{q}θ_{q}. The weights w_{q} are selected to minimize the variance of θᵢ under the assumption that θ_{q} are independent for different quartets. Thus, the optimal weights are determined by the following formula:

w_{q}=1/(σ_{q}²Σₚ1/σₚ²)

The estimated variance of θᵢ is

σᵢ²=1/Σ_{q}(1/σ_{q}²).

We have excluded from the further analysis 255,435 SNPs, for which numbers of available samples for each genotype were not sufficient to accurately determine described parameters; we required at least three samples with AA and BB genotypes and at least five samples with AB genotype. We further excluded 5,007 SNPs with high standard deviation of allele dosage (σᵢ>0.07) and 48,707 SNPs with significant deviation (p-value <0.05) from Hardy-Weinberg equilibrium. Since SNPs in strong linkage disequilibrium (LD) may result in false LOH regions, we excluded all SNPs in total LD with other SNPs as well as all SNPs for which deviation from total LD has been observed only in one sample. The total number of SNPs excluded due to LD was 115,438 (from each group of SNPs in LD we left a single SNP with the lowest variance σᵢ). In all number of SNPs excluded from the analysis was 424,607 out of total 500,568 SNPs. For the remaining 75,961 SNPs the median frequency of the minor allele is 34.4% and median standard deviation of allele dosage is σ=0.0298.

### Results

LOH due to heterozygous deletions for cell lines as well as tumor samples have been detected using our method of determination of CN values (see Example 2 below). To be able to detect LOH within copy number neutral and amplified regions within cell lines and tumor samples we have designed a different approach even though we still used results of our CN analysis to determine both CN values as well as contamination with benign tissue for tumor samples.

LOH Analysis in Cell Lines

Let us assume that we know copy number cᵢ at each SNP i. This information can be derived from copy number analysis described, e.g., in Example 2 below. LOH pattern of a sample can be described by assigning a variable h(i) to each SNP i such that h(i)=1 if SNP i is inside an LOH region and h(i)=0 otherwise. The corresponding likelihood can be written as

L{h(i)}=Πᵢ((Pᵢ(hom|θᵢ)fᵢ)^{h(i)}(Pᵢ(hom|θᵢ)fᵢ²+2Pᵢ(het|cᵢ,θᵢ)fᵢ(1−fᵢ))^{(1-h(i))}Πᵢ(Δ(h(i+1),h(i))+γ(1−Δ(h(i+1),h(i)))) (1)

Here the first product is taken over all SNPs, fᵢ is population frequency of allele A if θᵢ<=0.5 or allele B otherwise. It is assumed that alleles at neighboring SNPs are independent. This approximation is reasonable since SNPs in strong linkage disequilibrium are excluded (see Methods). The second product is taken over all pairs of adjacent SNPs, Δ(h(i+1),h(i))=1 if h(i+1)=h(i) and Δ(h(i+1),h(i))=0 otherwise, and log(γ)=−10 is the probability that one of the adjacent SNPs has LOH while the other SNP does not. The probability that SNP i is homozygous is estimated by

Pᵢ(hom|θᵢ)=exp(−(θᵢ−θ₀)²/(2σᵢ²))/(2π)^{1/2}σᵢ) (2)

where θ₀=0 if θᵢ<=0.5 and θ₀=1 otherwise. The probability a SNP i is heterozygous is estimated by

Pᵢ(het|cᵢ,θᵢ)=maxₙ{exp(−(θᵢ−n/cᵢ)²/(2σᵢ²))/((2π)^{1/2}σᵢ)} (3)

where maximum is taken over the number of copies n of allele B (0<n<cᵢ). The most likely LOH solution is the one that maximizes the likelihood L{h(i)}. The likelihood defines Hidden Markov Model (HMM) with the states being CNs of individual SNPs (Fridlyand et al., J. MULTIVAR. ANAL. (2004) 90:132-153). Therefore one can use forward-backward procedure (Fridlyand et al.) to find the maximum likelihood state. Since for high copy number regions, the difference in allele dosage θᵢ between a homozygous genotype and the genotype with only one copy of the alternative allele is very small, we restricted the analysis of cell lines to regions with copy numbers below 5 (cᵢ<5). In addition, regions with copy number below two (cᵢ<2) are excluded from analysis since regions with one copy always have LOH, and the regions with zero copies have undefined LOH status. In order to account for spurious outliers in allele dosage measurements we added a small value of 0.01 to the probabilities defined by equations (2) and (3).

This method was applied to the Affymetrix 500K GeneChip™ arrays data obtained for 178 cancer cell lines. There were large enough numbers of cell lines to analyze three types of cancer separately: breast (44 cell lines), colon (35 cell lines), and brain (19 cell lines). The rest of the cancer cell lines 80 were from 15 different cancers (14 ovarian cancer, 14 lung cancer, 13 melanoma, 11 leukemia, seven pancreatic cancer, six bladder cancer, three kidney cancer, two uterus cancer, two testicular cancer, two prostate cancer, two lymphoma, one thyroid cancer, one salivary gland cancer, one retina cancer, and one plasmacytoma) and were analyzed together. Results of the analysis averaged over the whole genome are presented in Table 1 with the exception of X chromosome for males.

TABLE 1

| | | | |
|---|---|---|---|
| | Fraction of | LOH for | Fraction of |
| | genome | this CN | total LOH |
| CN | (%) | (%) | (%) |

Breast cancer cell lines (44)

| | | | |
|---|---|---|---|
| 0 or above 4 | 10.08 | — | — |
| 1 | 4.17 | 100 | 13.09 |
| 2 | 34.42 | 55.98 | 60.51 |
| 3 | 29.94 | 19.39 | 18.23 |
| 4 | 21.39 | 12.17 | 8.17 |

Colon cancer cell lines (35)

| | | | |
|---|---|---|---|
| 0 or above 4 | 3.12 | — | — |
| 1 | 2.24 | 100 | 11.39 |
| 2 | 57.12 | 23.10 | 67.25 |
| 3 | 27.88 | 12.13 | 17.23 |
| 4 | 9.64 | 8.39 | 4.12 |

Brain cancer cell lines (19)

| | | | |
|---|---|---|---|
| 0 or above 4 | 1.71 | — | — |
| 1 | 4.78 | 100 | 21.12 |
| 2 | 54.78 | 24.21 | 58.67 |
| 3 | 30.95 | 12.34 | 16.90 |
| 4 | 7.79 | 9.60 | 3.31 |

80 cell lines from 15 different cancers

| | | | |
|---|---|---|---|
| 0 or above 4 | 3.74 | — | — |
| 1 | 4.93 | 100 | 19.96 |
| 2 | 52.45 | 29.05 | 61.71 |
| 3 | 27.97 | 12.34 | 13.98 |
| 4 | 10.91 | 9.83 | 4.34 |

100 ovarian tumor samples

| | | | |
|---|---|---|---|
| 0 or above 3 | 16.50 | — | — |
| 1 | 11.93 | 100 | 41.76 |
| 2 | 48.44 | 26.84 | 45.53 |
| 3 | 23.12 | 15.71 | 12.71 |

Table 1 shows the fraction of the genome occupied by regions with different copy numbers, frequency of LOH in these regions, as well as fraction of LOH accounted by regions with different copy numbers. The results are very similar for different cancers even though the cell lines were derived from tumors with different stage and grade. One can make several interesting observations. First, for all cancer cell lines LOH occurs predominantly in copy number neutral regions rather than in regions with heterozygous deletions. Second, the assumption (e.g., in Goransson et al.) that LOH rarely occurs in amplified regions is incorrect. According to our data, LOH happens as frequently in regions with copy number three as in regions with heterozygous deletions. While LOH in regions with copy number four is not the most important (mainly because these regions are not that frequent), it also accounts for a significant fraction of total LOH. Finally, regions with copy numbers above four are relatively rare and small and, therefore, they do not usually account for a significant fraction of total LOH.

Table 2 presents frequency of LOH in the cell lines by chromosome. While for some chromosomes the frequency is strongly dependent on the type of cancer cell lines, for other chromosomes the frequency of LOH is similar across different cancers. For example, on chromosomes 2, 7, and 20 frequency of LOH is relatively low, while on chromosomes 14, 22, 17, and X it is relatively high.

TABLE 2

| | Breast | Colon | Brain | 80 CLs | OvCa |
|---|---|---|---|---|---|
| | cancer | cancer | cancer | from 15 | tumor |
| | cell lines | CLs | CLs | diff. | samples |
| Chromosome | (CLs) (44) | (35) | (19) | cancers | (100) |
| 1 | 35.15 | 8.83 | 17.22 | 23.22 | 20.12 |
| 2 | 26.79 | 6.84 | 8.55 | 13.55 | 14.03 |
| 3 | 32.70 | 20.14 | 15.13 | 19.15 | 16.84 |
| 4 | 38.40 | 29.18 | 19.70 | 26.88 | 46.52 |
| 5 | 30.62 | 27.82 | 17.65 | 24.90 | 37.57 |
| 6 | 33.70 | 26.17 | 27.57 | 25.23 | 41.89 |
| 7 | 23.81 | 11.56 | 5.32 | 13.95 | 20.23 |
| 8 | 29.41 | 26.88 | 11.66 | 20.15 | 26.49 |
| 9 | 37.07 | 21.92 | 32.37 | 37.33 | 31.80 |
| 10 | 32.91 | 6.29 | 71.58 | 32.32 | 22.31 |
| 11 | 32.02 | 19.91 | 35.05 | 25.72 | 35.02 |
| 12 | 30.46 | 12.69 | 8.45 | 20.67 | 20.82 |
| 13 | 56.74 | 10.72 | 28.09 | 38.40 | 54.93 |
| 14 | 38.97 | 34.65 | 51.72 | 28.32 | 35.77 |
| 15 | 29.45 | 17.09 | 7.79 | 21.60 | 36.06 |
| 16 | 27.17 | 8.63 | 12.51 | 28.22 | 45.17 |
| 17 | 63.90 | 34.74 | 45.77 | 33.60 | 84.19 |
| 18 | 38.55 | 55.38 | 31.45 | 27.35 | 38.93 |
| 19 | 32.87 | 18.22 | 20.13 | 24.72 | 32.81 |
| 20 | 24.84 | 4.69 | 4.20 | 20.43 | 17.15 |
| 21 | 27.87 | 17.00 | 16.46 | 21.44 | 37.99 |
| 22 | 51.53 | 26.81 | 37.31 | 46.94 | 72.60 |
| X | 51.99 | 51.80 | 56.31 | 65.64 | 47.19 |

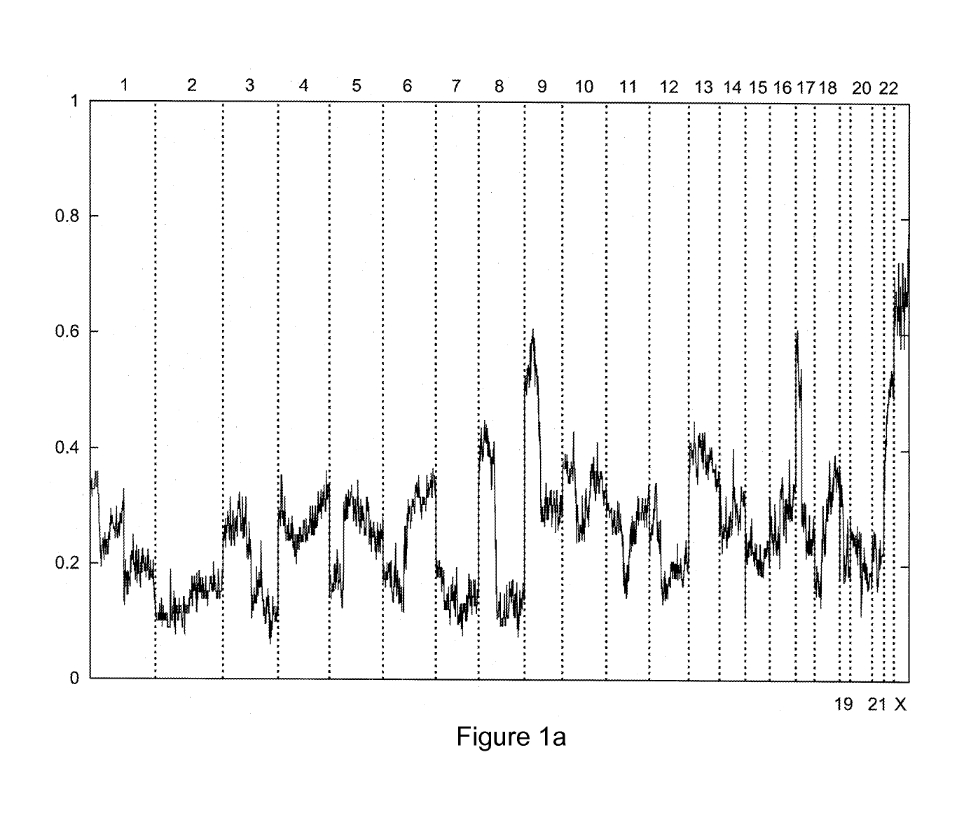

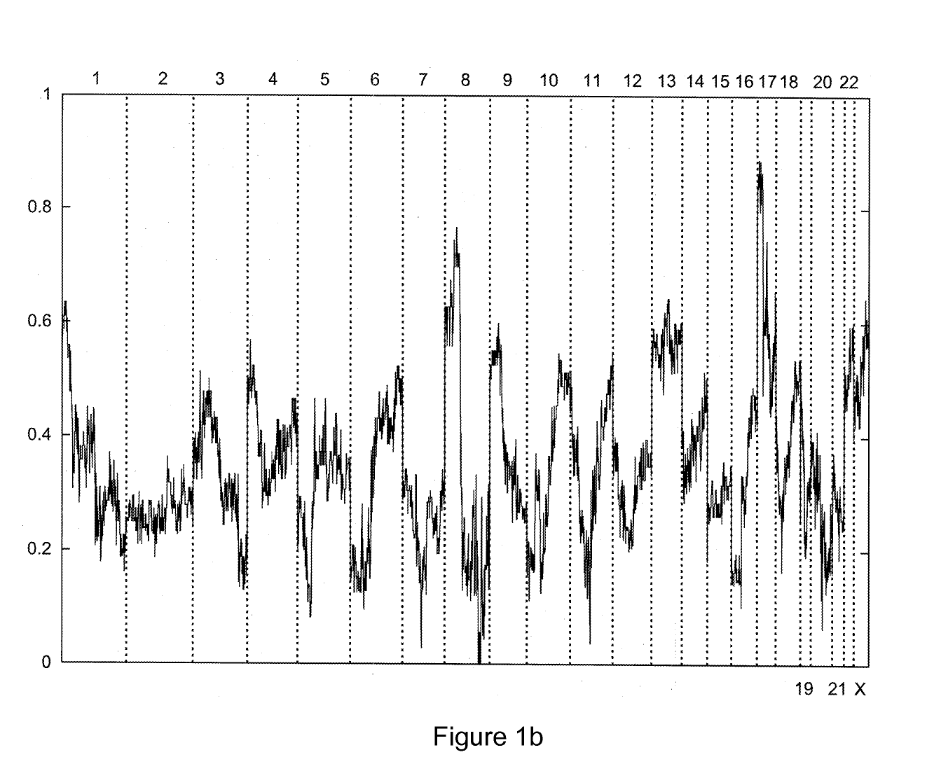

FIG. 1 shows LOH frequency across the whole genome. For 80 cell lines from 15 different cancers (FIG. 1a), the two highest LOH frequency peaks are on chromosome 9 covering the genes CDKN2A (p16) and CDKN2B (LOH ˜57%) and on chromosome 17 covering the TP53 gene (LOH ˜63%). For 44 breast cancer cell lines (FIG. 1b) the peak of LOH frequency on chromosome 8 covers four members of the tumor necrosis factor receptor superfamily: TNFRSF10A, TNFRSF10B, TNFRSF10C, and TNFRSF10D (LOH ˜74%). There are also two peaks with high LOH on chromosome 17 covering the genes TP53 (LOH ˜89%) and BRCA1 (LOH ˜73%). For colon cancer cell lines (data not shown in FIG. 1) we observed a sharp peak on chromosome 3 (LOH ˜79%) covering the FHIT gene, another peak on chromosome 17 covering the TP53 gene (LOH ˜57%), and a wide region of high LOH (˜71%) on chromosome 18. For brain cancer cell lines (data not shown in FIG. 1) the highest LOH peaks are on chromosome 9 covering the genes CDKN2A (p16) and CDKN2B (LOH ˜71%), on chromosome 10 covering the entire chromosome (LOH ˜79%), and on chromosome 17 covering the TP53 gene (LOH ˜68%). These results are summarized in Table 3 below.

TABLE 3

| | | |
|---|---|---|
| | | LOH |
| Chromosome | Genes | Freq. |

Breast Cancer

| | | |
|---|---|---|
| 8 | TNFRSF10A, | 74% |
| | TNFRSF10B, | |
| | TNFRSF10C, | |
| | TNFRSF10D | |
| 17 | TP53 | 89% |
| 17 | BRCA1 | 73% |

Brain Cancer

| | | |
|---|---|---|
| 9 | CDKN2A, | 71% |
| | CDKN2B | |
| 10 | n/a | 79% |
| 17 | TP53 | 68% |

Colon Cancer

| | | |
|---|---|---|
| 3 | FHIT | 79% |
| 17 | TP53 | 57% |
| 18 | n/a | 71% |

Other Cancers

| | | |
|---|---|---|
| 9 | CDKN2A, | 57% |
| | CDKN2B | |
| 17 | TP53 | 63% |

LOH Analysis in Tumors

Similar to the analysis of cell lines described above, we assume that copy number cᵢ at each SNP i is known. In addition we assume that the degree of contamination a with normal DNA is also known. The likelihood of a particular LOH pattern can be written as

L{h(i)}=Πᵢ((Pᵢ(hom|θᵢ)fᵢ²+Pᵢ(hom,het|cᵢ,θᵢ)fᵢ(1−fᵢ))^{h(i)}(Pᵢ(hom|θᵢ)fᵢ²+2Pᵢ(het,het|cᵢ,θᵢ)fᵢ(1−fᵢ))^{(1-h(i))}Πᵢ(Δ(h(i+1),h(i))+γ(1−Δ(h(i+1),h(i)))) (4)

The probability that SNP i is homozygous in normal DNA (and therefore in cancerous DNA as well) is given by equation (2). The probability that SNP i is heterozygous in normal DNA but homozygous in cancerous DNA due to LOH is given by

Pᵢ(hom,het|cᵢ,θᵢ)=exp(−(θᵢ−θ₀(cᵢ,α))²/(2σᵢ²))/((2π)^{1/2}σᵢ) (5)

where θ₀(c,α)=α/((1−α)c+2α) if θᵢ<0.5 and θ₀(c,α)=((1−α)/((1−α)c+2α) otherwise.

The probability that SNP i is heterozygous in both cancerous and normal DNA is given by

Pᵢ(het,het|cᵢ,θᵢ)=maxₙ{exp(−(θᵢ−θₙ(cᵢ,α))²/(2σᵢ²))/((2π)^{1/2}σᵢ)} (6)

where θₙ(c,α)=((1−α)n+α)/((1−α)c+2α) and n is the number of copies of allele B (0<n<cᵢ). Since for high copy number regions, the difference in allele dosage θᵢ between a homozygous cancerous genotype and the genotype with only one copy of the alternative allele in tumors contaminated with normal cells is even smaller than in cell lines, we restricted the analysis of tumors to regions with copy numbers below 4 (cᵢ<4). A small value of 0.01 is added to the probabilities defined by equations (2), (5), and (6) to account for spurious outliers in allele dosage measurements.

This method was applied to the Affymetrix 500K GeneChip™ array data obtained for 165 ovarian tumor samples. To obtain more reliable data we excluded from further analysis 45 samples with a high level of noise and 20 samples with non-tumor cell contamination above 55%. The median contamination of the remaining 100 samples with normal cells was 24%. Stage, grade, and residual size after debulking surgery for these tumors are presented in Table 4.

TABLE 4

| | | Fraction |
|---|---|---|
| Parameters | Value | (%) |
| Stage | 1 | 8 |
| | 2 | 9 |
| | 3 | 70 |
| | 4 | 17 |
| Grade | 1 | 7 |
| | 2 | 15 |
| | 3 | 78 |
| Residual | 0 | 9.5 |
| tumor size | 1 | 61 |
| | 2 | 29.5 |

Results of LOH analysis averaged over the whole genome are presented in Table 1 above. Similar to cancer cell lines, in tumors LOH occurs predominantly in copy number neutral regions and regions with three copies. LOH frequency by chromosomes is presented in Table 2 above. We observe strikingly high frequency of LOH for chromosomes 17 (up to 88%) and 22 (up to 84%). See FIG. 1c.

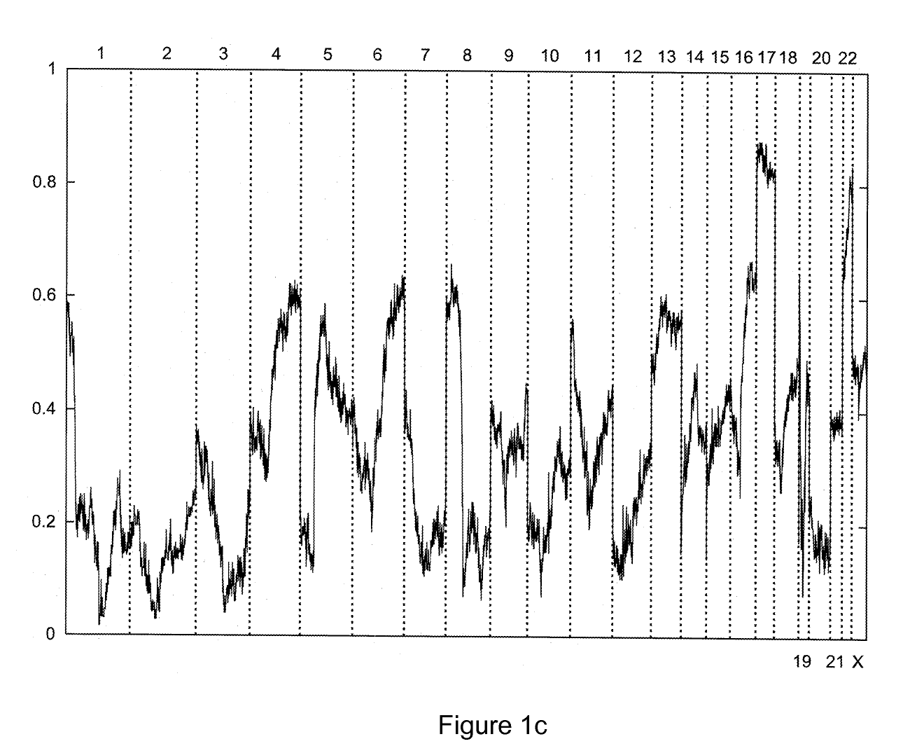

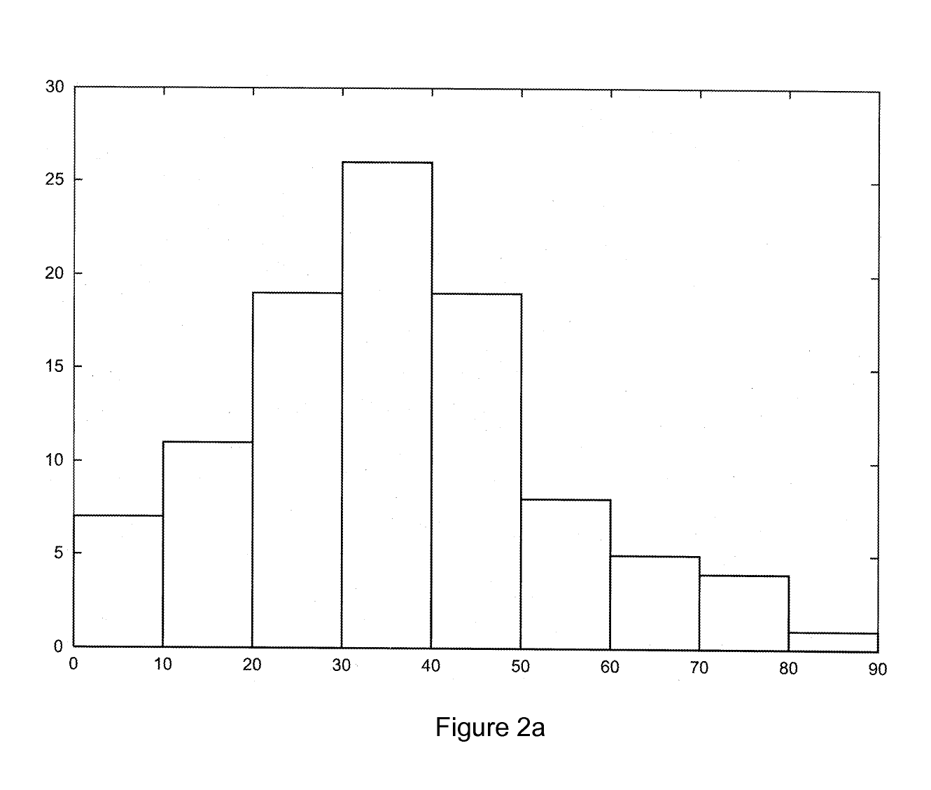

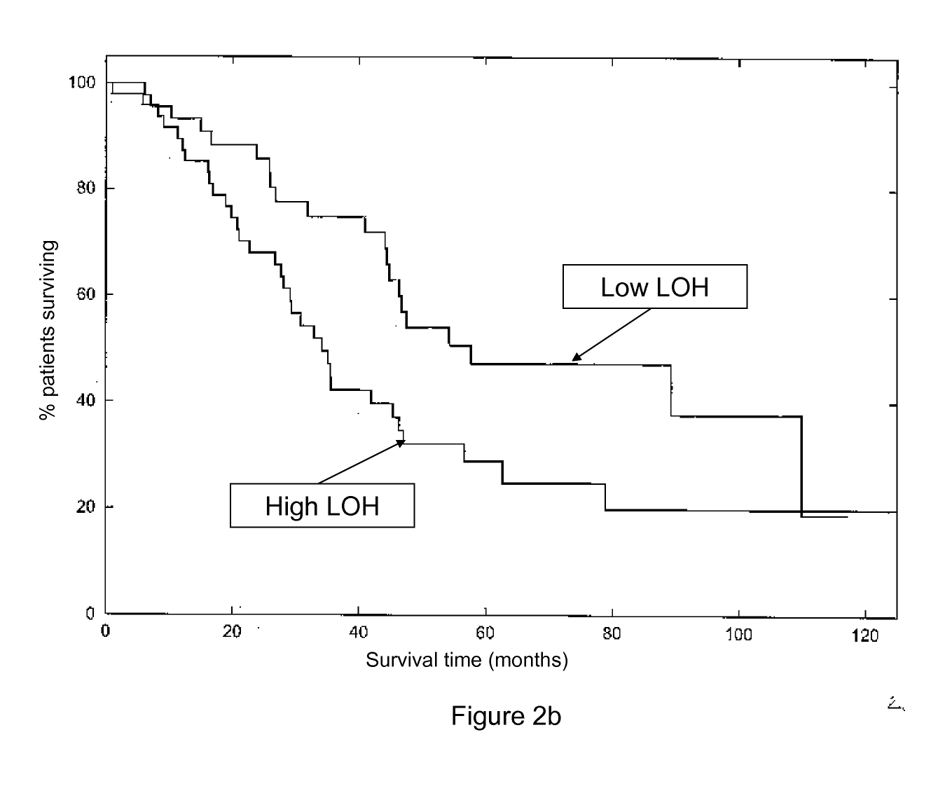

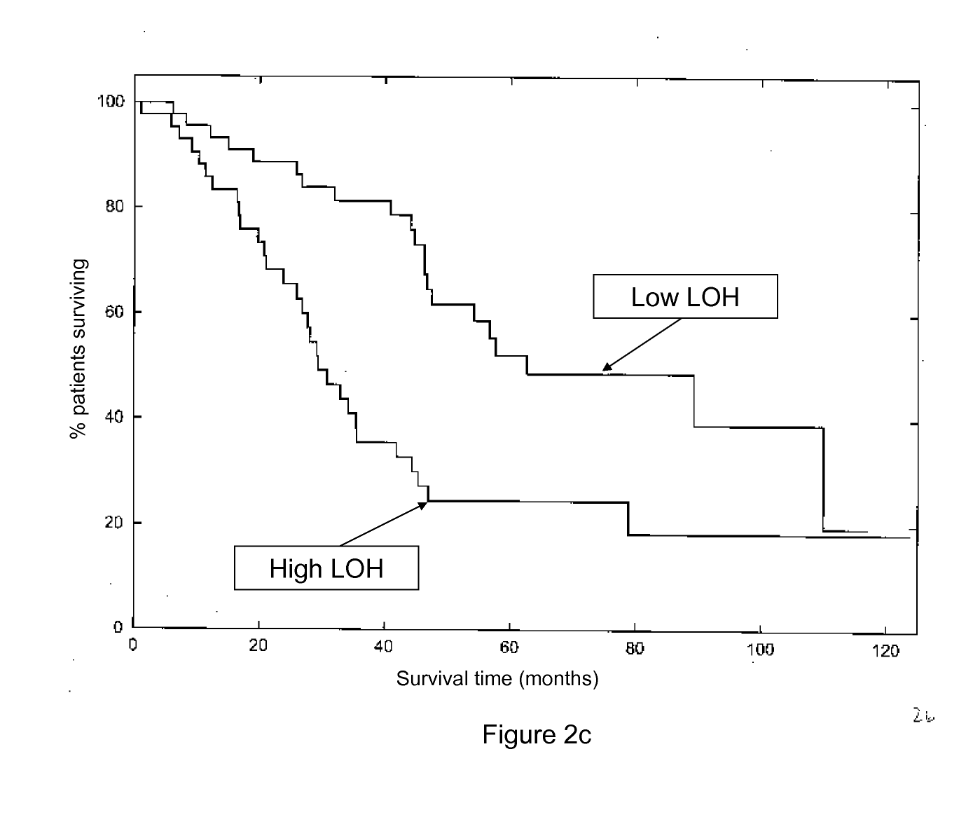

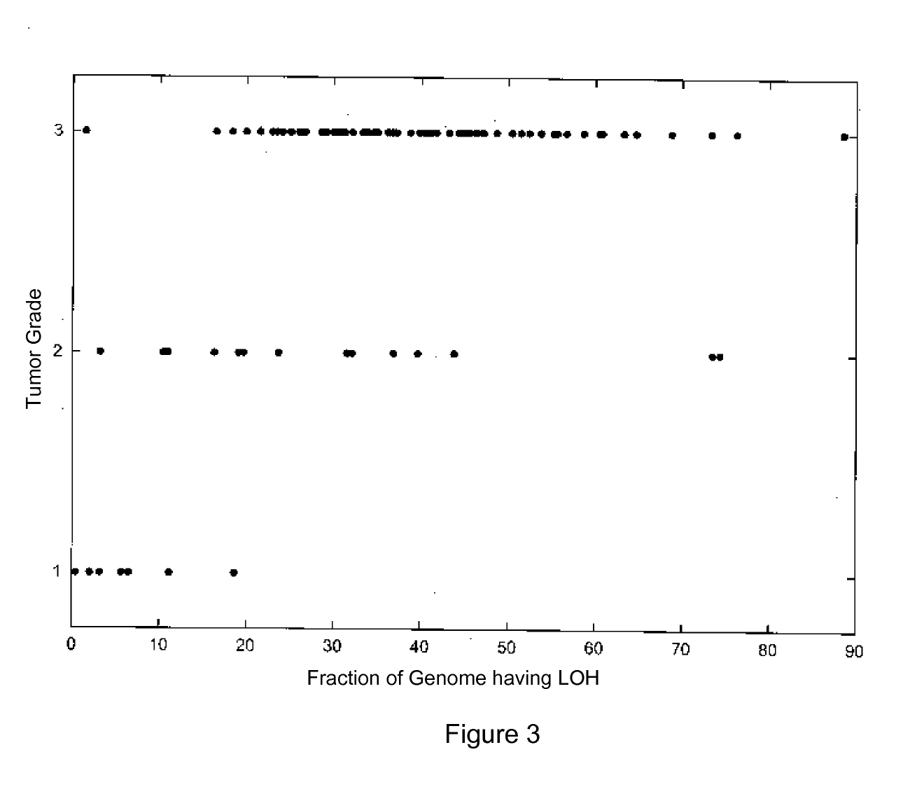

For each sample we calculated the proportion of overall LOH, also called fraction of the genome with LOH (“FGLOH”), using regions with copy number from one to three. FIG. 2a shows the distribution of FGLOH over samples. The range of FGLOH is quite wide, from 0.4% to almost 90%, with the median value of 35%. We investigated how FGLOH correlates with available clinical data: stage, grade, and residual size of the tumor after debulking surgery. We did not observe any statistically significant correlation between FGLOH and either stage or residual size of the tumor after debulking surgery. However we observed highly significant correlation with tumor grade (see FIG. 3) with correlation coefficient 0.48 and p-value=1.7×10⁻⁶. For grade 1 tumors median FGLOH is equal to 5.6%, for grade 2 tumors median FGLOH is equal to 23.7%, while for grade 3 tumors median FGLOH is equal to 73.9%.

We have also studied the association between FGLOH and overall survival of patients with ovarian cancer. Using the median amount of LOH (35%) as the cut-off between high and low LOH, the association has been evaluated using a Cox proportional hazards model for time to death from cancer after surgery. The results of our statistical analysis are combined in Table 5 below.

TABLE 5

| | | Number | |
|---|---|---|---|
| | | of | |
| Type of analysis | Parameters | samples | p-value |
| Univariate | Tumor stage | 97 | 0.043 |
| | Tumor grade | 97 | 0.12 |
| | Residual size | 92 | 0.00062 |
| | FGLOH | 97 | 2.80 × 10⁻⁵ |
| Multivariate | Tumor stage | 92 | 0.49 |
| | Tumor grade | 92 | 0.69 |
| | Residual size | 92 | 0.004 |
| | FGLOH | 92 | 0.0003 |

We found no significant association between overall survival of patients and tumor grade. Association with tumor stage was barely significant in univariate analysis and completely insignificant in multivariate analysis. However associations between overall survival of patients and FGLOH and residual size of tumor were significant in both univariate and multivariate analysis. To illustrate these effects we used Kaplan-Meier plots (FIG. 2). FIG. 2b presents a Kaplan-Meier plot of survival versus time after surgery for two groups of patients with ovarian cancer of the same size with FGLOH being the selection criteria. FIG. 2c presents a similar analysis with the exception that samples were divided into two groups using both FGLOH and the residual tumor size as the selection criteria. These parameters were weighted according to the results from the Cox model.

### Discussion

We have developed a novel computational method to determine LOH from SNP microarray data. The method can be used to analyze either cancer cell lines or tumor samples in the presence of significant contamination with normal cells. The method has been implemented for analysis of Affymetrix 500K GeneChip array data. The method requires a prior knowledge of copy number profile as well as the level of contamination with normal cells. This information can be obtained, e.g., using the methods discussed in Example 2 below.

We have applied the method to 178 cell lines representing multiple cancers as well as to 100 ovarian tumors. We observed that most LOH is copy number neutral LOH while LOH in regions with one copy represents only a small fraction of total LOH. We also surprisingly observed that LOH is quite common in amplified regions with copy number three or more. Another observation is that frequency of LOH strongly depends on chromosomal location. Some chromosomes, such as 17, have high frequency of LOH across many cancers. Other regions have high frequency of LOH only in certain cancers.

In a set of 100 ovarian tumors we investigated how FGLOH correlates with available clinical parameters. Specifically, we found that FGLOH correlates strongly with tumor grade and overall survival in both univariate and multivariate analysis. See, e.g., Table 5, FIG. 2.

### Example 2

We have developed a computational method capable of detecting regions with deletions and amplifications as well as estimating actual copy numbers in these regions. The method is based on determining how signal intensity from different probes is related to copy number (“CN”), taking into account changes in the total genome size, and incorporating into analysis contamination of the solid tumors with benign tissue. Hidden Markov Model is used to obtain the most likely CN solution. The method has been implemented for Affymetrix 500K GeneChip arrays and Agilent 244K oligonucleotide arrays. We have performed CN analysis for normal cell lines, cancer cell lines, and tumor samples. The method is capable of detecting copy number alterations in tumor samples with up to 80% contamination with benign tissue. Analysis of 178 cancer cell lines reveals multiple regions of common homozygous deletions and strong amplifications encompassing known tumor suppressor genes and oncogenes as well as novel cancer related genes.

Many known computational methods, e.g., those reviewed in Chari et al., CANCER INFORMATICS (2006) 2:48-58, detect regions with deletions and amplifications without estimating the actual CN in these regions. For example, these methods do not clearly distinguish between homozygous (CN=0) and heterozygous (CN=1) deletions which may have significantly different effects on cancer development; losing just one of two copies of a gene may not have as drastic of an effect as losing both copies. In fact, heterozygous deletions are very common and affect large regions including chromosomal arms and whole chromosomes. In contrast, homozygous deletions are much less common and usually affect small regions with only a few genes deleted. Similarly, it is important to be able to distinguish between low and high copy amplifications. Due to their overall genomic instability, cancer cells might accumulate multiple random CN changes which do not contribute to cancer development.

In this study we developed a method to estimate CN values for normal samples, cell lines, and solid tumors. To be able to do this we had to determine how signal intensity from different probes is related to CN, how to take into account change of the genome size in cancer cell lines and solid tumors which result from somatic alterations, and finally how to incorporate into the analysis contamination of the solid tumors with benign tissue. The last factor is of particular importance as solid tumor samples are strongly contaminated with non-cancerous cells carrying DNA without somatic alterations. Existing dissection techniques can reduce this contamination but cannot eliminate it entirely. For example, it is practically impossible to eliminate immune cell infiltration of tumors. In our experience, even ovarian tumors which are considered less affected by non-cancerous contamination than other tumors often contain more than 50% normal, non-cancerous, DNA. Such strong contamination presents a significant problem for estimating CN. For example, if in a tumor sample a particular region appears to have one copy, the actual CN in cancerous cells can be either one (CN=1) if the sample has negligible contamination or zero (CN=0) if the contamination is about 50%.

### Materials and Methods

Genomic DNA

Frozen tumors were cut into 10 μm sections and macrodissected to minimize contamination with normal tissue. A QIAamp DNA Mini Kit™ (QIAgen) was used to isolate the DNA as per the manufacturer's protocol with an overnight lysis incubation at 56° C., including the optional RNase A treatment. DNA was quantitated using a Nanodrop spectrophotometer and picogreen.

Affymetrix 500K GeneChip™ Arrays

The Affymetrix GeneChip Mapping™ NspI or StyI Assay Kit was used in the generation of biotinylated DNA for Affymetrix Mapping 500K™ NspI or StyI microarray hybridizations (each assay was prepared separately). Genomic DNA (250 ng) was digested with NspI or StyI restriction enzyme and adaptors were added to restriction fragment ends with T4 DNA ligase. Adaptor-modified samples were PCR™ amplified using Clontech Titanium Taq™ which generated an amplified product of average size between 200 and 1,100 bp. Amplification products were purified using a Clontech DNA amplification cleanup kit. 90 μg of purified DNA was fragmented using Affymetrix Fragmentation Reagent. Biotin-labeling of the fragmented sample was accomplished using the GeneChip DNA Labeling Reagent. Biotin-labeled DNA was hybridized on NspI or StyI Affymetrix microarrays at 49° C. for 16 to 18 hours in the Affymetrix rotation oven. After hybridization, probe array wash and stain procedures were carried out on the automatic Affymetrix Fluidics Stations as per manufacturer's manual and microarrays were scanned and raw data was collected by Affymetrix GeneChip Scanner 3000™.

Quantitative Real-Time PCR

12 cell lines were identified with copy number amplifications detected by microarray spanning the genes CCND1, EGFR and/or ERBB2. Custom qPCR assays (Applied Biosystems) were designed which would amplify a region of genomic DNA from each of these genes, and from 2 genomic regions where the cell lines did not show any copy number changes. These 2 assays were used as calibrators to calculate dCt in subsequent analyses. Genomic DNA from each of the cell lines was diluted to 5 ng/μl. 1 μl genomic DNA, 8 μl H₂O, 1 μl qPCR assay, and 10 μl TaqMan Universal PCR Master Mix™ (Applied Biosystems) were combined and cycled in an Applied Biosystems 7900™ real time PCR instrument using the following conditions: 50° C. for 2 mins, 95° C. for 10 mins, 40 cycles of 95° C. for 15 seconds, 60° C. for 1 minute, hold at 4° C.

Agilent 244K CGH ARRAYS

0.5-3 ug test and reference (Promega, p/n G152A) genomic DNA samples were simultaneously digested with AluI and RsaI restriction enzymes. Following fragmentation, the DNA samples were labeled using an Agilent Genomic Enzymatic Labeling Kit. The labeling kit uses random primers and the exo-Klenow fragment to label DNA through incorporation of fluorescently labeled nucleotides (Cy3-dUTP or Cy5-dUTP, for test or reference DNA respectively). Labeled samples were purified by Microcon YM-30 columns or AutoScreen-96A Well plates. The concentration of the purified samples was determined using a NanoDrop ND-1000 spectrophotometer.

Equal amounts of test and reference fluorescently labeled samples were pooled and heat denatured after being combined with cot-1 DNA, Agilent aCGH blocking agent and Agilent Hi-RPM hybridization solution. Microarray hybridizations were performed using Agilent SureHyb Hybridization chambers. Hybridization chambers were loaded onto a rotisserie in an Agilent Hybridization Oven and were incubated at 65° C. for 40 hours with a rotational speed of 20 rpm. Following incubation, the microarray slide was washed for 5 minute in aCGH/ChIP-on-chip Agilent Wash Buffer 1 at room temperature and 1 minute in Agilent Wash Buffer 2 at 37° C. Microarray slides were scanned on an Agilent scanner and raw data were collected.

### Results

Combining Signals from Multiple Probes on Affymetrix 500K GeneChip Arrays

The Affymetrix 500K GeneChip array contains 25-mer oligonucleotides distributed over two subarrays, Nsp1 and Sty1 containing 262,264 and 238,304 SNPs, respectively. Each SNP on the array is represented by either six or ten oligonucleotide probe quartets consisting of perfect-match (PM) and mismatch (MM) pairs for both SNP alleles. These probes reside on Nsp and Sty PCR amplicons, which range in size from 100 bp to 1143 bp. For CN analysis the signal intensities from the multiple probes representing a SNP should be optimally combined to generate a single value corresponding to the SNP.

It is known in the art that GC content and length of PCR amplicons affect signal intensities from probes and this effect varies from sample to sample. It also appears that the effect of amplicon size is much stronger than the effect of GC content. Our data (not shown) support this conclusion. Therefore, we did not take GC content into account. However, we made an adjustment for amplicon size. First for each quartet and allele, the intensity I_{MM} from the MM probe is subtracted from the intensity I_{PM} from the PM probe: ΔI=I_{PM}−I_{MM}. Then for a given amplicon size s, an average ΔI_{S} over all quartets and alleles for SNPs residing on PCR amplicons of size s within a subarray is calculated. Finally, a normalized intensity I_{qX}=ΔI/ΔI_{S} is calculated for each allele X and each quartet q where s is the size of the corresponding amplicon.

The next step is to combine signals from both alleles A and B within a quartet q using the following formula:

S_{q}=k_{A}I_{qA}+k_{B}I_{qB}

where k_{A} and k_{B} are normalization parameters. These parameters are introduced to ensure that S_{q} is close to 2 independent of the genotype (AA, AB, or BB) of a sample. The ratio of these parameters, k_{A}/k_{B}, represents uneven allele amplification described previously for SNP arrays. In order to estimate parameters k_{A} and k_{B}, we have used genotyping data for 48 cell lines provided by Affymetrix. Because these cell lines were collected from non-cancerous tissue we assume that for almost all SNPs copy number will be equal to 2. Therefore, for each SNP we sought to minimize the following expression:

E(k_{A},k_{B})=N_{AA}(k_{A}x_{AA}+k_{B}y_{AA}−2)²+N_{AB}(k_{A}x_{AB}+k_{B}y_{AB}−2)²+N_{BB}(k_{A}x_{BB}+k_{B}y_{BB}−2)².

Here N_{XX} is the number of samples with genotype XX for this SNP among 48 samples (N_{AA}+N_{AB}+N_{BB}=48), while x_{XX} is the median value of I_{qA} in samples with genotype XX and y_{XX} is the median value of I_{qB} in these samples. Minimizing the function E(k_{A},k_{B}) over parameters k_{A} and k_{B} results in the following formulae:

k_{A}=2(CₓC_{yy}−C_{y}C_{xy})/(CₓₓC_{yy}−C_{xy}C_{xy})

k_{B}=2(C_{y}Cₓₓ−CₓC_{xy})/(CₓₓC_{yy}−C_{xy}C_{xy})

where

Cₓ=N_{AA}x_{AA}+N_{AB}x_{AB}+N_{BB}x_{BB}

C_{y}=N_{AA}y_{AA}+N_{AB}y_{AB}+N_{BB}y_{BB}

C_{xy}=N_{AA}x_{AA}y_{AA}+N_{AB}X_{AB}y_{AB}+N_{BB}x_{BB}y_{BB}

Cₓₓ=N_{AA}x_{AA}x_{AA}+N_{AB}x_{AB}x_{AB}+N_{BB}x_{BB}x_{BB}

C_{yy}=N_{AA}y_{AA}y_{AA}+N_{AB}y_{AB}y_{AB}+N_{BB}y_{BB}y_{BB}.

Using these formulae we calculated parameters k_{A} and k_{B} for all SNPs on Affymetrix 500K GeneChip arrays except for 10,841 SNPs for which all 48 cell lines were homozygous. These SNPs were excluded from the analysis. To evaluate the strength of the uneven allele amplification effect we calculated the median of max(k_{A}/k_{B},k_{B}/k_{A}). The median is about 1.40, meaning that for a typical SNP on Affymetrix 500K GeneChip arrays, signal intensity for one of the alleles is about 40% higher than the signal intensity for the other allele.

The values S_{q} for all quartets for SNP i are linearly combined with weights reflecting their performance: Sᵢ=Σ_{q}w_{q}S_{q}. The weights are selected to minimize the variance of Sᵢ under the assumption that the deviations of S_{q} from 2 are independent for different quartets. Thus, the optimal weights are determined by the following formula:

w_{q}=(1/σ_{q}²)/Σₚ(1/σₚ²)

where σ_{q}² is the variance of S_{q} within 48 cell lines. The estimated variance of Sᵢ is

σᵢ²=1/Σ_{q}(1/σ_{q}²).

To evaluate the importance of optimization of weights for different quartets, we can compare σᵢ² with the corresponding variance resulting from using equal weights for all quartets. The estimate for the latter variance is sᵢ²=(1/nᵢ)Σ_{q}σ_{q}², where nᵢ is the number of quartets for SNP i. Median ratio of the variances, sᵢ²/σᵢ², over all SNPs is about 1.97 which means that for a typical SNP using optimal weights should reduce variance almost two fold.

For each SNP we have calculated σᵢ² defined as (Sᵢ−2)² averaged over 48 cell lines. The median σᵢ² for all SNPs is 0.0376. We have excluded 1,832 SNPs with σᵢ² exceeding an arbitrary cutoff 0.25. The important exception of the described method concerns SNPs on the X chromosome (outside the XY region). For these SNPs parameters were determined using only cell lines from females.

Some of the PCR amplicons on the Affymetrix 500K GeneChip array contain more than one SNP. Any CN changes affecting such an amplicon should equally affect all SNPs within the amplicon. Therefore, signals Sᵢ for all SNPs within an amplicon are averaged and assigned to one of the SNPs while the other SNPs are excluded from further analysis. This reduces the number of SNPs by 82,189 leaving 405,706 SNPs for CN analysis.

Relationship Between SNP Signal Intensity and CN

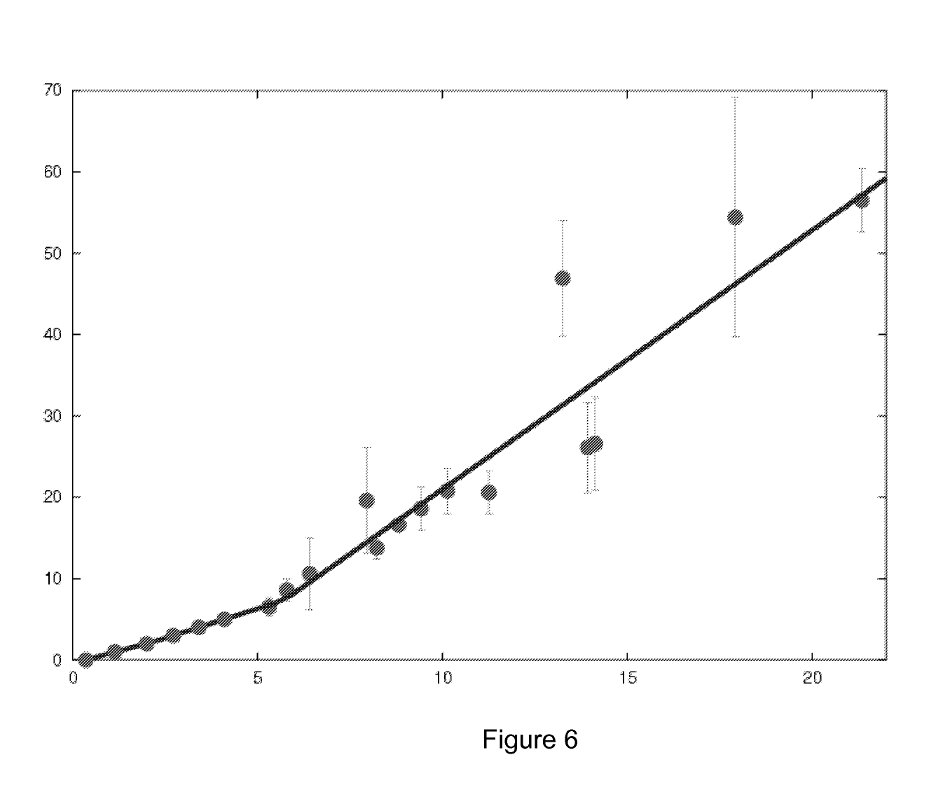

While normalized SNP signal intensities Sᵢ described in the previous section are supposed to generate an average signal of 2.0 for SNPs within regions of genome with CN=2, one should not expect that the average signal is equal to the actual CN in the regions of the genome where CN is different from two. For example, the slope of the relationship between signal intensity from an array and the amount of DNA in the solution differs from the ideal value 1.0. Moreover, this relationship might be nonlinear as well. To evaluate this effect for regions of the genome with CN=1 we used SNPs on the X chromosome (outside the XY region) for 28 cell lines from males provided by Affymetrix. In addition, we have analyzed cell lines with multiple X chromosomes: NA04626 (XXX), NA01416E (XXXX), and NA06061 (XXXXX) This allowed us to estimate the relationship between CN and signal intensity up to CN=5. Finally, we have selected a few cancer cell lines for which it has been reported that certain genes are strongly amplified. These cell lines and a reference sample RS were analyzed by qPCR assays amplifying the corresponding genes as well as a reference gene RG which has 2 copies in these cell lines. CN for the amplified genes were estimated using the formula CN=exp(−C_{Tgc}+C_{Trc}−C_{Tgr}+C_{Trr}), where C_{Txy} is the C_{T} value for gene x (x=g for an amplified gene and x=r for the reference gene) in sample y (y=c for a cell line and y=r for the reference sample). The same cell lines were run on Affymetrix 500K GeneChip arrays. Comparison of the results allowed us to estimate the relationship between CN and signal intensity for high CN values. Results of the experiments are shown in Table 6 and FIG. 6. Signal intensity for SNPs in regions with CN>8 increases roughly linearly with CN. Therefore, for CN c the expected signal intensity S₀(c) is given by Table 6 for c<8 and S₀(c)=0.314c+3.41 for c>7.

TABLE 6
Median signal intensities.

| Cell line | Median signal | Comment |
|---|---|---|
| PC3 | 0.36 | Homozygous deletion of PTEN |
| Data for 28 male cell | 1.14 | X |
| lines | | |
| provided by Affymetrix | | |
| NA04626 | 2.72 | XXX |
| NA01416E | 3.41 | XXXX |
| NA06061 | 4.1 | XXXXX |
| HTB27 | 5.31 | CN estimated by qPCR for CCND1 is 6.5 ± 1.1 |
| CRL1620 | 5.79 | CN estimated by qPCR for EGFR is 8.6 ± 1.4 |
| CCL251 | 6.41 | CN estimated by qPCR for CCND1 is 10.6 ± 4.4 |
| HTB25 | 8.22 | CN estimated by qPCR for CCND1 is 13.8 ± 1.4 |
| CRL2338 | 8.82 | CN estimated by qPCR for CCND1 is 16.6 ± 0.7 |
| HTB27 | 9.42 | CN estimated by qPCR for ERBB2 is 18.6 ± 2.7 |
| CRL1978 | 7.95 | CN estimated by qPCR for CCND1 is 19.6 ± 6.5 |
| HTB23 | 11.26 | CN estimated by qPCR for CCND1 is 20.6 ± 2.6 |
| HTB19 | 10.13 | CN estimated by qPCR for EGFR is 20.8 ± 2.8 |
| HTB127 | 13.93 | CN estimated by qPCR for CCND1 is 26.1 ± 5.5 |
| CRL2321 | 14.13 | CN estimated by qPCR for CCND1 is 26.6 ± 5.7 |
| HTB41 | 13.25 | CN estimated by qPCR for CCND1 is 46.9 ± 7.1 |
| HTB128 | 17.92 | CN estimated by qPCR for CCND1 is 54.4 ± 14.7 |
| HTB127 | 21.34 | CN estimated by qPCR for ERBB2 is 56.5 ± 3.9 |

Adjustment for Average CN and Contamination with Normal DNA

As described above, one of the steps in deriving S involves normalization by average intensity of other SNPs. This normalization allows accurate estimation of CN for non-cancerous samples where most of the genome has two copies. In cancerous samples the average CN, due to amplifications and deletions, can be very different from two and, therefore, additional signal normalization is required. In addition, tumor samples are often contaminated with non-cancerous cells with most of the genome having two copies. As a result, the expected signal intensity S(c) for a SNP within regions with CN c is defined by the following equation:

S(c)=β(S₀(2)α+(1−α)S₀(c))=β(2α+(1−α)S₀(c)) (1).

Here β is a normalization factor to account for change in average CN within the genome of cancerous cells, and α is the degree of contamination of tumor samples with normal cells. This equation is applicable to all types of samples: normal non-cancerous cells, cancer cell lines, and tumors. However, for cancer cell lines we assume no contamination with non-cancerous cells and, therefore, α=0. For non-cancerous samples we assume that the average CN is equal to 2.0 and, therefore, β=1 and α=0.

In order to estimate the parameters α and β, we use the following algorithm. First, for each SNP i we calculate smoothed signal Tᵢ:

log(Tᵢ)=(1/N)Σⱼ log(Sⱼ)

where the sum is taken over N=101 SNPs surrounding SNP i (j=i−50, . . . , i+50). Then we calculate the histogram H(T) of the smoothed signals with bin size of 0.1. The histogram is expected to have local maxima corresponding to CN values present in a given sample. Let n be the total number of the local maxima and Tₖ be the positions of the maxima (k=1, . . . , n). For a given pair of parameters α and β, for each maximum k we assign an integer CN value cₖ which minimizes the absolute difference |log(Tₖ)−log(S(cₖ))| where S(c) is given by equation (1). Then we calculate the following sum:

E₁(α,β)=ΣₖH(Tₖ)(log(Tₖ)−log(S(cₖ)))² (2).

This sum measures how well a given pair of parameters α and β fits the positions of all the maxima Tₖ. In order to find the best parameters α₀ and β₀, we minimize E₁(α,β) by direct enumeration of all pairs of α and β with α within the interval (0, 1) and β within the interval (0.5, 2). Unfortunately, this minimization is not always able to distinguish between the correct solution and incorrect ones. In order to avoid incorrect solutions, before optimization over parameters α and β we assign CN cₖ to some of the maxima Tₖ according to the following rules:
1. If there is only one maximum (n=1), then C₁=2.
2. If n>1 and the first maximum has SNP genotyping call rate below 60%, then c₁=0.
3. If n>1 and the first maximum has SNP heterozygosity below 3%, then c₁=1.
4. If n>2, c₁=0 and the second maximum has SNP heterozygosity below 3%, then c₁=1.
5. If n>1 and the highest maximum is at least two times higher than the second highest maximum, then the highest maximum is assigned CN=2.

Rule 2 is based on the assumption that the SNP genotyping call rate should be low in the regions with CN=0. Rules 3 and 4 are based on the assumption that SNP heterozygosity should be low in the regions with CN=1. Both assumptions are valid for cell lines and tumors with low contamination with non-cancerous DNA. Rules 1 and 5 are based on the assumption that the dominating CN=2.

Hidden Markov Model

The likelihood that SNPs have copy numbers cᵢ can be written as

L{cᵢ}Πᵢ exp(−(log(Sᵢ)−log(S(cᵢ)))²/(2σ²))Πᵢ(Δ(cᵢ₊₁,cᵢ)+γ(1−Δ(cᵢ₊₁,cᵢ)) (3).

Here the first product is taken over all SNPs, and the normal distribution is assumed for deviation of the natural logarithm of the actual SNP signal Sᵢ form the natural logarithm of the expected signal S(cᵢ). In addition, the deviations are assumed to be independent and to have the same standard deviation σ. The second product is taken over all pairs of adjacent SNPs, Δ(cᵢ₊₁, cᵢ)d=1 if cᵢ₊₁=cᵢ and Δ(cᵢ₊₁, cᵢ)=0 otherwise, and γ is the probability that the adjacent SNPs have different CNs. The most likely CN solution is the one that maximizes the likelihood L{cᵢ}. The most likely solution depends both on σ and γ, or more precisely, on the value of −σ² log(γ).

The likelihood defines Hidden Markov Model (HMM) with the states being CNs of individual SNPs. Therefore one can use forward-backward procedure to find the maximum likelihood state. In order to make the model computationally tractable, we limit the largest possible CN to be 70. Another simplification is related to the fact that individual chromosomes are independent and, therefore, the maximization of the likelihood is performed separately for all chromosomes. First, we find the maximum likelihood state using values for α and β obtained by minimization of the expression (2). Then we minimize over α and β the following sum:

E₂(α,β)=(1/N)Σᵢ(log(Sᵢ)−log(S(cᵢ)))² (4).

Then we find the maximum likelihood state using values for α and β obtained by minimization of the expression (4). We repeat this iteration procedure until it converges as defined by change in α upon one iteration being less than 3% and change in β being less than 0.1%. Formula (4) also provides the estimate of the variance σ² in expression (3).

Analysis of 48 Normal Cell Lines

First we have analyzed the data for 48 normal cell lines provided by Affymetrix. One of the advantages of this analysis is that the samples were collected from families (triplets), allowing us to test our algorithm; germ line deletions and amplifications should be inherited from parents. In addition, CN changes are expected to be relatively uncommon. Using this dataset we have optimized parameter γ in formula (3) to reduce the number of false positive and false negative CN changes: log(γ)=54. We have found multiple amplifications with CN up to five and heterozygous deletions (CN=1) as well as a few very small homozygous deletions. These observations are consistent with published results. As expected, we have found CN=1 for X chromosome for all males. Moreover, for all males we have observed CN=2 for the XY region.

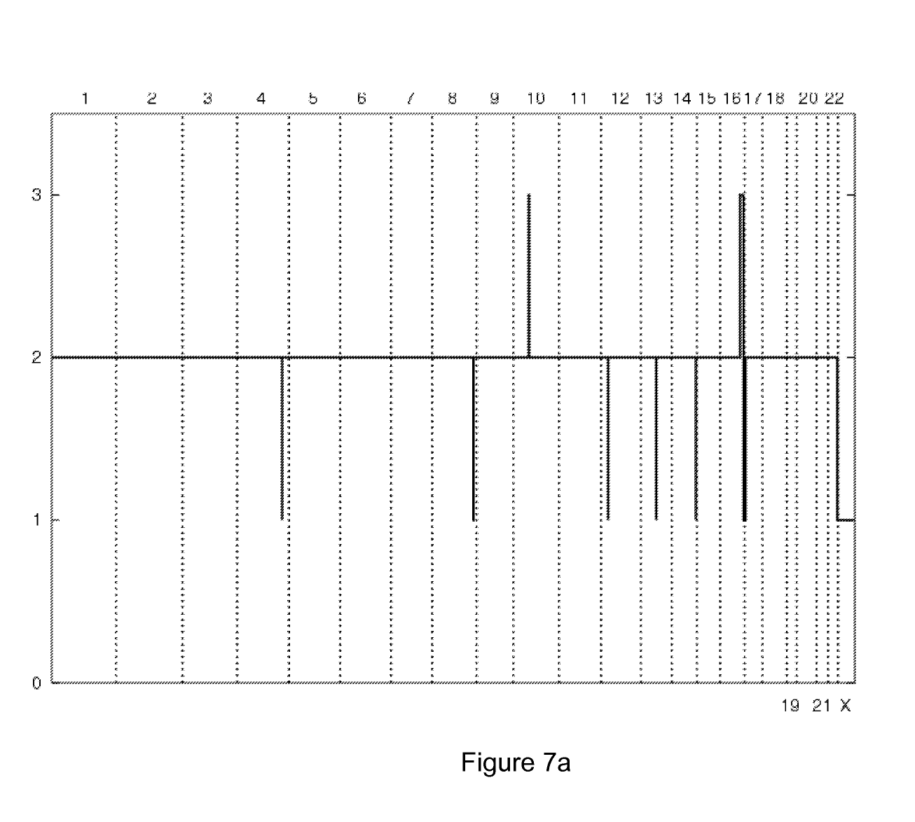

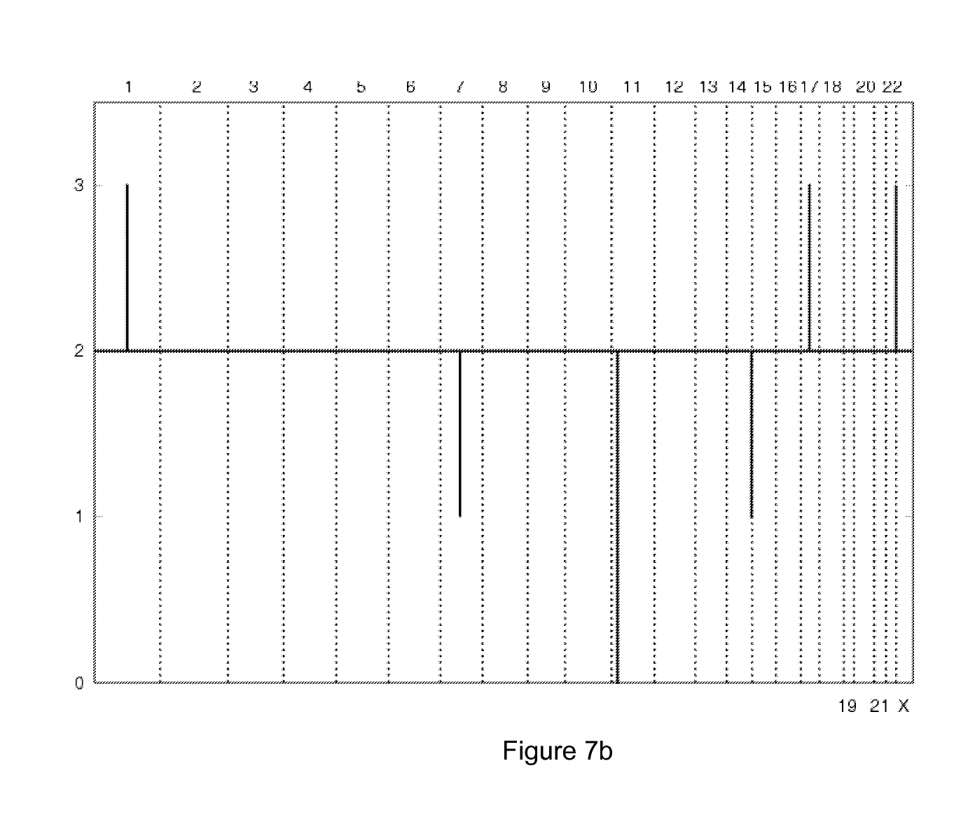

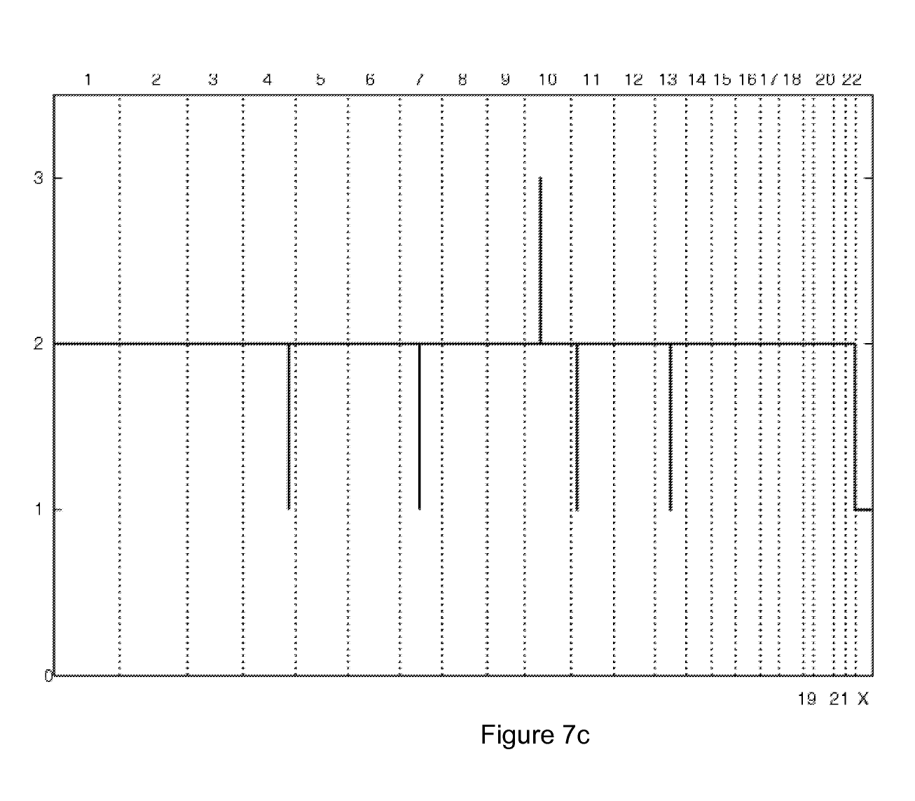

Results of the analysis of one of the triplets (NA12056 from the father, NA12057 from the mother, and NA10851 from the son) are shown on FIG. 7. All deletions and amplifications in NA10851 are, as expected, inherited from the parents: heterozygous deletion on chromosome 4 from the father, heterozygous deletion on chromosome 7 from the mother, amplification to CN=3 on chromosome 10 from the father, heterozygous deletion on chromosome 11 from the mother (who actually has a homozygous deletion of this region), and heterozygous deletion on chromosome 13 from the father.

Similar analysis of the other triplets revealed some incompatibilities between CN alterations in children and their parents. Some of these incompatibilities are caused by true somatic CN alterations in the samples as was reported previously. Other incompatibilities are caused by the algorithm either missing some real alterations (false negatives) or reporting some unreal alterations (false positives). We found that the rate of false alterations is high for alterations encompassing only a few SNPs. Starting from seven SNPs the rate of false CN alterations is below 5%.

Analysis of 178 Cancer Cell Lines

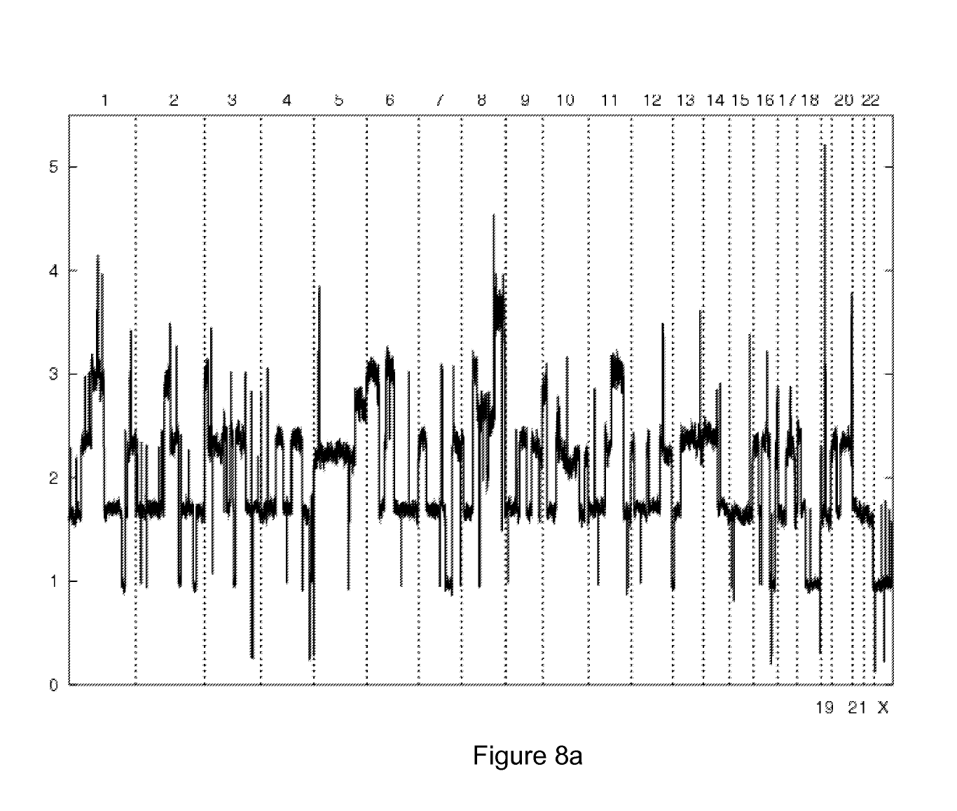

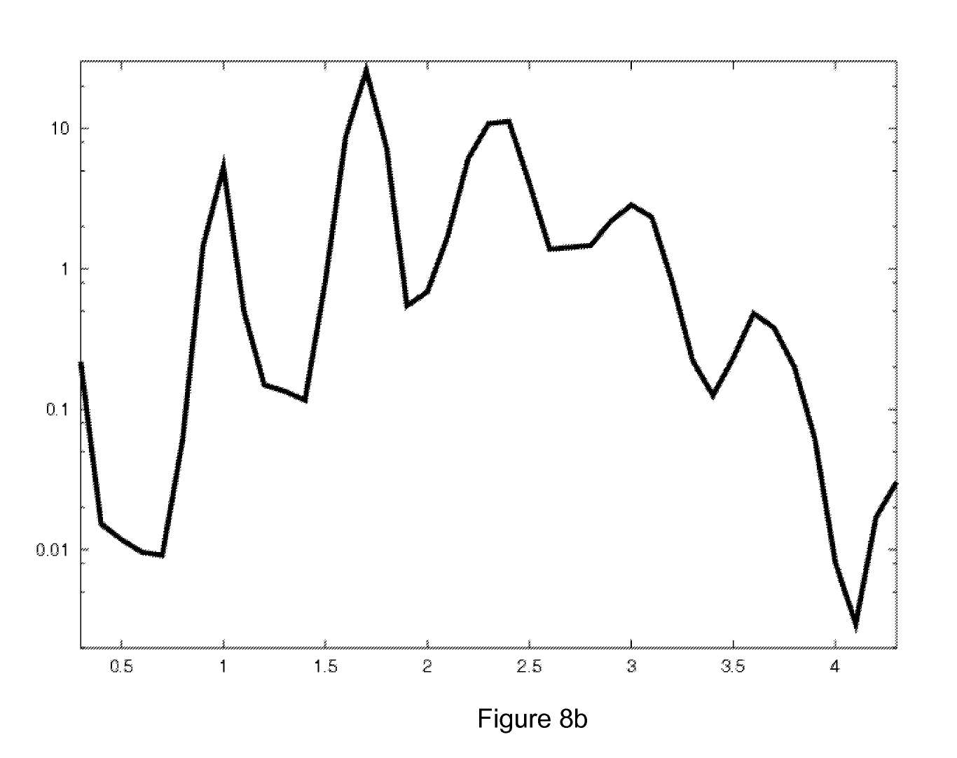

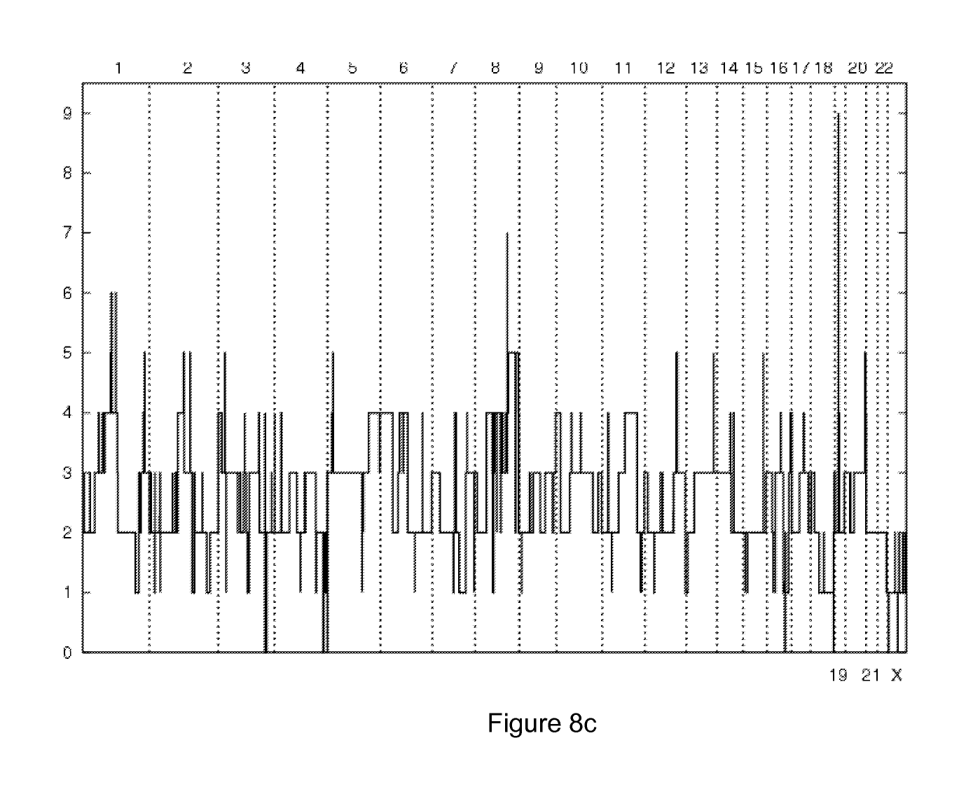

As an example we present here the analysis of the ovarian cancer cell line OVCAR8. Smoothed signals Tᵢ are shown in FIG. 8a with the corresponding histogram shown in FIG. 8b. There are seven maxima (n=7) in the histogram. Table 7 lists call rates and heterozygosities for SNPs within the seven maxima as well as heights H(Tₖ) of the maxima. SNPs within the first maximum have low call rate and thus are assigned CN=0. SNPs within the second peak have low heterogeneity and thus are likely to correspond to CN=1. The third peak has to correspond to CN=2, which is supported by the fact that this peak is the highest. Since this is a cell line, we assume that α=0. Minimization of E₁(α=0, β) over β gives β=1.18. After adjustment it becomes clear that the fourth peak corresponds to CN=3, the fifth to CN=4, the sixth to CN=5, and the seventh to CN=6. Running HMM refines the estimate for β=1.17. The final result of CN analysis for this cell line is presented on FIG. 8c. Homozygous deletions of several genes are observed including EVI1 and WWOX.

TABLE 7
Heterozygosity and call rate of SNPs within seven
p maxima of signal intensity for ovarian cancer
cell line OVCAR8 (see FIG. 8b).

| Position of a | SNP | Call rate | Height |
|---|---|---|---|
| maximum | heterozygosity (%) | (%) | (%) |
| 0.3 | 9.1 | 36.7 | 0.2 |
| 1.0 | 1.2 | 90.2 | 5.3 |
| 1.7 | 8 | 88.1 | 25.8 |
| 2.4 | 14.2 | 82 | 11.2 |
| 3.0 | 19 | 77.6 | 2.8 |
| 3.6 | 1 | 98.4 | 0.5 |
| 4.3 | 28.9 | 69.6 | 0.03 |

We have analyzed 178 different cancer cell lines (44 breast, 35 colon, 19 brain, 14 ovarian, 14 lung, 13 melanoma, 11 leukemia, seven pancreatic, six bladder, three kidney, two uterus, two testicular, two prostate, two lymphoma, one thyroid, one salivary gland, one retina, and one plasmacytoma). The number of CN variations in these cell lines varied from two to 369 (the median value being 94). We have observed 510 homozygous deletions encompassing seven or more SNPs. Homozygous deletions, which were observed in at least five different cancer cell lines, are shown in Table 8. Some of these deletions encompass well known tumor suppressor genes such as p16 and PTEN. Others might encompass unknown tumor suppressor genes, and some of these deletions might simply happen within fragile parts of the genome. We have also observed 580 amplifications with CN of at least 10. Those amplifications, which were observed in at least five different cancer cell lines, are shown in Table 9. Some of these amplifications encompass well known oncogenes such as MYC and ERBB2. Others might encompass oncogenes which are not yet known.

TABLE 8
Frequent homozygous deletions observed in 178 cancer cell lines.
Chromosomal positions are based on March 2006 version of the
UCSC Human Genome Browser.

| | Number of | Likely tumor suppressor |
|---|---|---|
| Region | observations | gene |
| chr2: 141238933-141834821 | 7 | LRP1B |
| chr3: 59686057-61195849 | 29 | FHIT |
| chr4: 91615328-92272605 | 6 | |
| chr6: 162091010-162697695 | 5 | PARK2 |
| chr7: 38238069-38371496 | 6 | |
| chr7: 141707985-142210594 | 5 | |
| chr9: 19866496-28550130 | 36 | CDKN2A (p16), CDKN2B |
| chr10: 88921717-91164155 | 6 | PTEN |
| chr14: 21159134-22067364 | 7 | |
| chr16: 6161277-7101949 | 9 | |
| chr16: 76814181-77773689 | 26 | WWOX |
| chr20: 14275899-15352425 | 22 | |

TABLE 9
Frequent amplifications with CN > 9 observed in 178 cancer
cell lines. Chromosomal positions are based on March 2006
version of the UCSC Human Genome Browser.

| | Number of | |
|---|---|---|
| Region | observations | Likely oncogene |
| chr8: 81240524-81974370 | 5 | TPD52 |
| chr8: 113091665-114742342 | 10* | |
| chr8: 128756816-128849113 | 17 | MYC |
| chr8: 142078709-142107781 | 7** | PTK2 |
| chr11: 68619900-69985448 | 11 | CTTN, FGF4, FGF3, |
| | | FGF19, ORAOV1, |
| | | CCND1, MYEOV |
| chr17: 34999505-35264341 | 14 | ERBB2 |
| chr17: 43477124-44795465 | 10 | |
| chr17: 55677511-61149311 | 6 | APPBP2, PPM1D, BCAS3 |
| chr20: 45554593-46054338 | 5 | NCOA3 |
| chr20: 55503756-56241375 | 5 | |
| chr20: 57622421-58603945 | 6 | |
| chr22: 19057363-20130955 | 5 | CRKL |

*One of these amplifications also involves MYC.
**Two of these amplifications also involve MYC.

Adjustment on Contamination with Benign Tissue in Tumor Samples

In order to check how well our method estimates the degree of contamination α of tumors with benign tissue, we artificially contaminated DNA samples of eight cancer cell lines (see Table 10) from different tissues with DNA extracted from CEPH cell line NA12776. All cancer cell lines as well as NA12776 were sampled from female subjects. The DNA samples were quantitated by picogreen three times for higher precision and then combined to give degrees of contamination of the different cancer cell lines with NA12776 DNA between 10% and 80%. After that we ran these mixed samples on Affymetrix microarrays and estimated degrees of contamination using our algorithm. Results of this analysis as well as the mix ratio based on DNA quantitation are presented in Table 10. For the majority of samples the difference between the estimated α and the DNA quantitation data were within a few percent. This is quite remarkable, because the observed difference is close to the error rate of the picogreen quantitation. The observed differences tend to be higher for higher contamination levels.

TABLE 10
Comparison of the degree of contamination of cancer cell
lines with CEPH cell line NA12776 determined using the
picogreen quantitation (averaged over three measurements)
and the algorithm presented in this paper.

| | | | | Percent of |
|---|---|---|---|---|
| | | | | SNPs with |
| | | | | concordant CN |
| | | | | values for |
| | | Mix ratio from | Mix ratio | contaminated vs |
| Cancer cell | Tissue | DNA | from CN | pure cancer |
| line | type | quantitation | analysis | cell lines |
| HTB19 | Breast | 10 ± 1% | 9.6% | 97.1% |
| HTB76 | Ovary | 20 ± 4% | 18.2% | 98.6% |
| HTB127 | Breast | 30 ± 4% | 33.6% | 98.5% |
| CCL228* | Colon | 40 ± 3% | 43.4% | 95.9% |
| CCL253* | Colon | 50 ± 2% | 60.7% | 88.2% |
| HTB119 | Lung | 60 ± 2% | 54.1% | 99.8% |
| HTB9 | Urinary | 70 ± 2% | 74.3% | 97.0% |
| | bladder | | | |
| HTB41 | Salivary | 80 ± 1% | 84.7% | 56.9% |
| | gland | | | |

*For these two samples one of the picogreen measurements failed.

The cancer cell lines used in the mixing experiment were also run on Affymetrix microarrays without any mixing. Comparison between results of CN analysis of mixed versus non-mixed samples is presented in Table 10. The concordance between CN results is expressed as percent of SNPs with the same CN. With one exception the concordance is above 95% for contamination levels below 80%. While the sensitivity of CN analysis inevitably decreases as contamination with benign tissue increases (and small alterations in CN affecting only a few SNPs become indistinguishable from noise) our algorithm is able to determine the degree of contamination as well as major changes in copy number correctly in strongly contaminated samples.

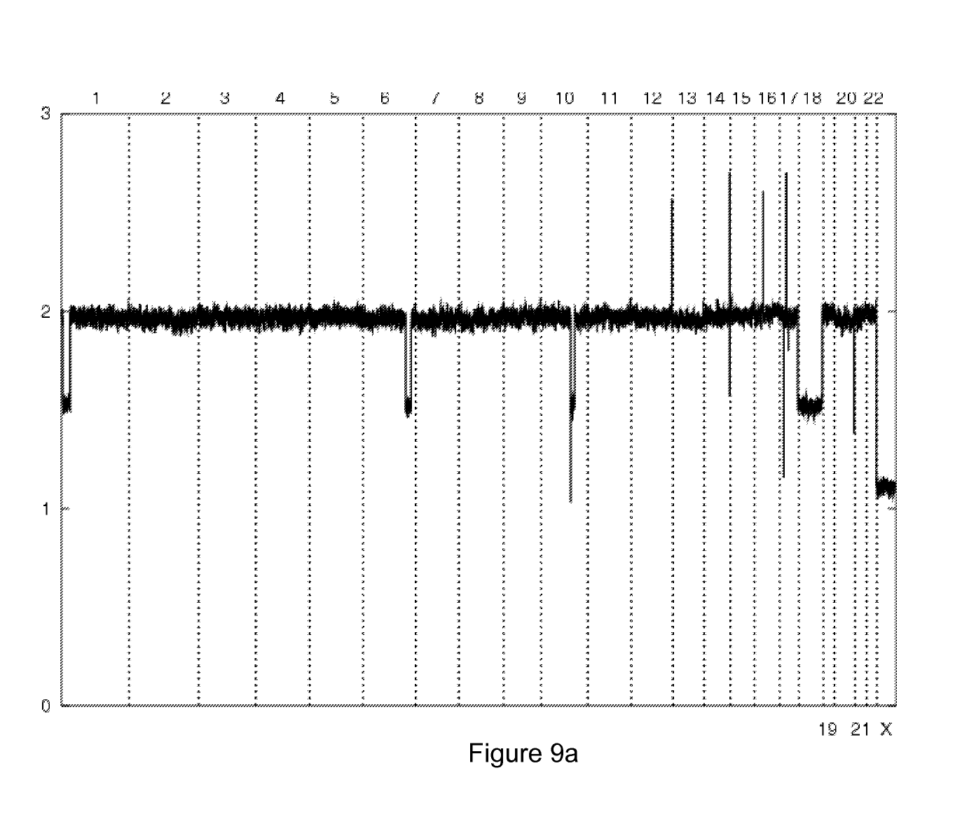

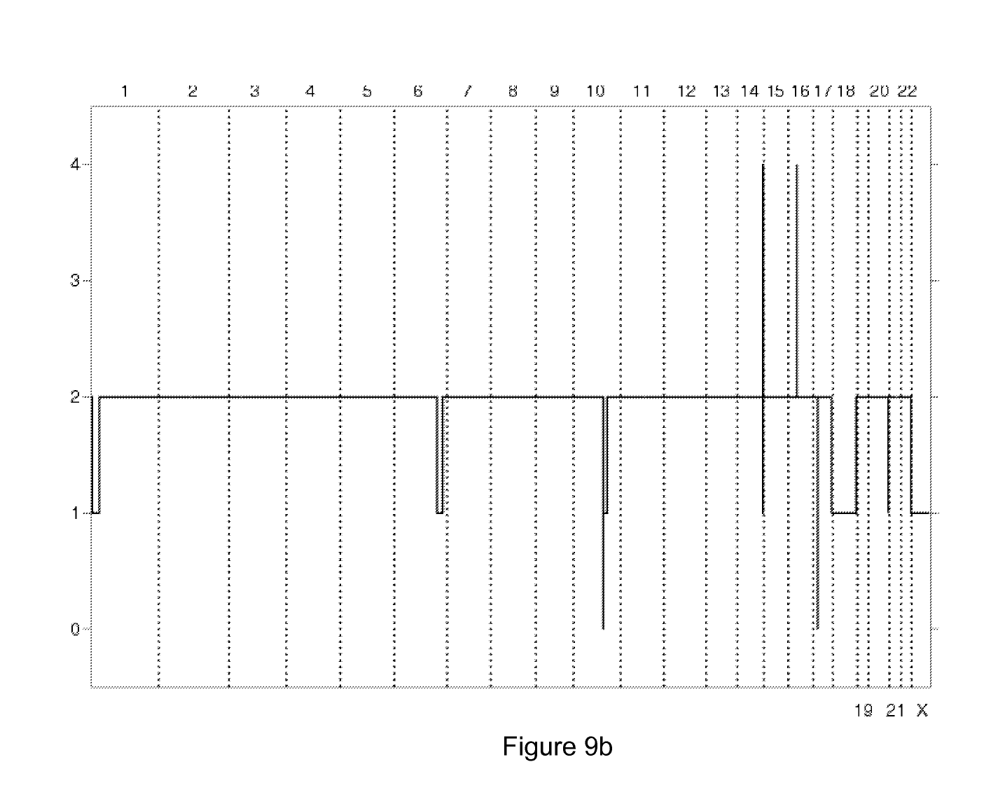

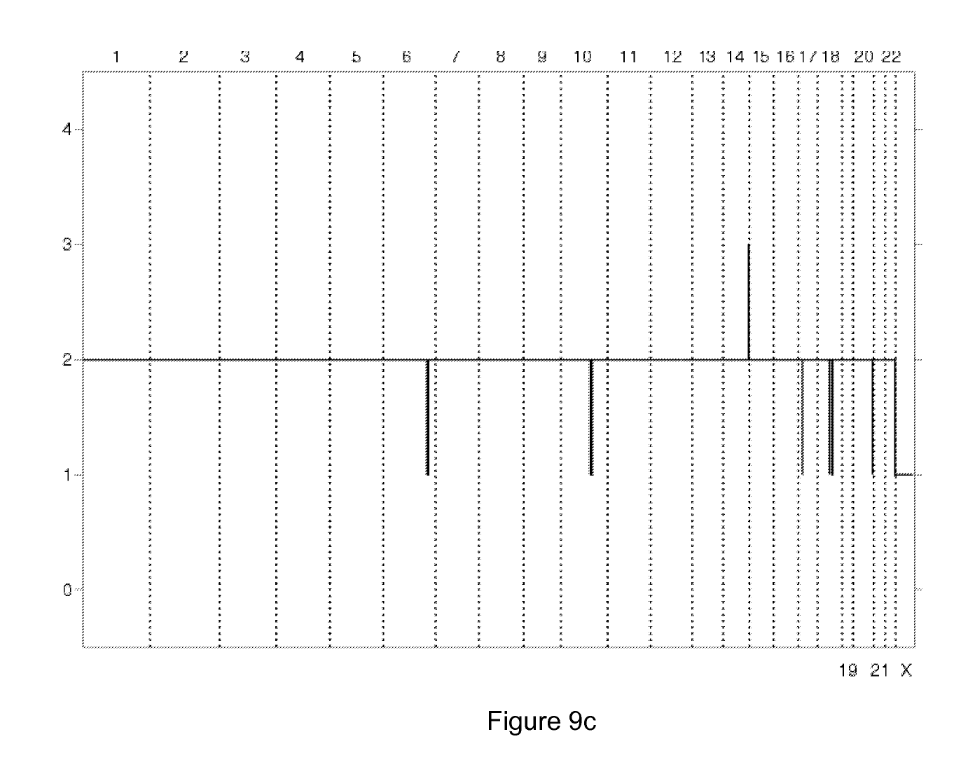

FIG. 9 demonstrates the importance of adjustment on the contamination with benign tissue for CN analysis of tumor samples. In FIG. 9a one can see signal intensities of SNPs for a colon tumor sample. The contamination of this sample with benign tissue, determined by our program, is about 48%. Such strong contamination leads to a dramatic shift of levels of signal intensities for SNPs within regions with different CN values. The right CN solution after adjustment of signal intensity on contamination with benign tissue is presented on FIG. 9b. Because this sample was collected from a male subject there is only one copy of X chromosome in both tumor and normal cells. As a result, despite contamination the signal intensity for SNPs within the X chromosome (outside the XY region) is the same as it should be for SNPs within regions with CN=1 in the absence of any contamination. However, one can see that the signal intensity of SNPs within large heterozygous deletions on chromosomes 1, 6, 10, and 18 is significantly higher than the signal intensity of SNPs within the X chromosome (see FIG. 9a). SNPs within amplified regions on chromosomes 13, 15, 16, and 17 have signal intensity as if their CN=3. However, after adjustment on contamination it becomes clear that these are actually amplifications to CN=4. Moreover, SNPs within homozygous deletions on chromosome 10 and 17 because of the contamination appear to have the same signal intensity as SNPs within the X chromosome. In FIG. 9c we presented the CN solution, obtained by the HMM algorithm, if adjustment on contamination with benign tissue is not made. All CN variations in this solution have been determined incorrectly (besides single copy of the X chromosome): heterozygous deletions on chromosomes 1 and 15 and amplification on chromosome 16 are lost altogether, positions for the heterozygous deletions on chromosomes 6, 10, 18, and 21 are wrong, homozygous deletions on chromosome 10 (gene PTEN) and on 17 are instead called heterozygous, and, finally, CN=3 is assigned for the amplification on chromosome 15 instead of CN=4. Generally we have been able to determine CN values in solid tumors even when the degree of contamination with benign tissue is significant.

Using Agilent 244K Oligonucleotide Arrays for Copy Number Analysis

Agilent 244K oligonucleotide arrays with complete genome coverage are designed for copy number analysis. Instead of SNP probes the array has 60-mer oligonucleotide probes. Our method of analysis of Agilent array data is very similar to the described method of analysis of Affymetrix SNP array data with only two differences. First, we do not need to combine signals from individual probes into a SNP signal, rather we use signal intensities supplied by Agilent software. Second, when we estimate α and β in equation (2), we cannot use either SNP call rate or SNP heterozygosity.

### Discussion

We developed a new method of CN analysis of the data from Affymetrix 500K GeneChip arrays and Agilent 244K oligonucleotide arrays. The method is designed to determine positions of CN variations as well as to estimate actual CN. In most of the published algorithms, authors attempt to determine position of CN variations and distinguish between deletions and amplifications without determining the actual value of CN. We reason that estimating actual CN is extremely important. For example, it is likely that most CN variations with CN=1, 3, and 4 are random in the sense that they are not positively selected for during cancer development. On the other hand, homozygous deletions and strong amplifications are most likely to be detected in the regions with genes related to cancer cell survival and growth.

In order to be able to estimate actual CN within amplified and deleted regions, we have determined how signal intensity from different probes is related to CN, how to take into account change of the genome size in cancer cell lines and solid tumors as the result of somatic alterations, and finally how to incorporate into the analysis contamination of the solid tumors with the benign tissue. HMM has been used to determine exact CN values as well as the positions of the affected regions.

We have tested our method on 48 normal cell lines derived from nuclear families. We have detected a number of germ line CN alterations with proper inheritance from parents to children. We also applied our algorithm to 178 cancer cell lines derived from different tissues. We identified multiple regions with common homozygous deletions and high level amplifications. As expected some of these regions harbor well known oncogenes and tumor suppressor genes. Other regions do not encompass well known cancer related genes. Then we applied our algorithm to artificial mixes of DNA derived from cancer cell lines with DNA derived from normal cell lines. These mixes model contamination of tumor samples with non-cancerous cells. The estimates of the contamination of cancer DNA with normal DNA produced by our algorithm for these mixes matched closely the corresponding experimental estimates using DNA quantitation by picogreen. Comparison between CN alterations detected by our algorithm in the mixed samples versus pure samples showed very high concordance in particular for contamination levels below 50%. Finally, using as an example a colon cancer tumor sample with about 50% contamination with normal DNA, we have demonstrated how erroneous results of CN analysis can be without properly taking into account the effect of the contamination.

One of the potential limitations of the presented method is related to the assumption that all cancer cells within a cell line or a tumor sample have the same CN profile. This assumption seems to be correct for most of the cell lines and tumor samples we have analyzed. However, some samples (˜10%) are clearly heterogeneous; not all the cells in these samples share all CN alterations. For such samples our method may at times produce erroneous results within the regions not shared by all cancer cells. Fortunately, these regions represent only a fraction (˜5%) of the genome and, therefore, do not impact significantly the results for the rest of the genome.

Another limitation of the method is the assumption of independence of signal noise for closely positioned probes (see Equation (3)). However, it is reasonable to expect that the noise for closely positioned probes should be correlated. We do observe such correlation but the correlation appears to be negligible for Affymetrix 500K SNP arrays and Agilent 244K oligonucleotide arrays. On the other hand, we observed a very strong correlation for adjacent probes on custom Agilent arrays with extremely dense coverage of specific regions (data not shown). This correlation strongly affects the performance of our algorithm and, most likely, other published algorithms; in particular, multiple false CN alterations may be observed on such custom, densely-packed arrays. It would be advantageous to modify the presented algorithm to properly take into account this correlation for arrays with dense coverage.

### Example 3

We have also found a correlation between overall LOH levels and progression-free survival in breast cancer. Specifically, we analyzed a publicly available database study, Gene Expression Omnibus (GEO) accession no. GSE10099, that provides data for 97 ER positive breast cancer samples. The experimental details for this dataset, including microarray details, are available on the NCBI website at http://www.ncbi.nlm.nih.gov/geo/query/acc.cgi?acc=GSE10099.

After running Affymetrix's “Genotyping Console 3.0.2” software on this dataset's CEL files to generate genotyping calls, we applied the same statistical and computational analysis from Examples 1 & 2 above to the dataset. We found that the amount of LOH in breast cancer patients is correlated with poor prognosis—i.e., decreased progression-free survival (p-value=0.015).

### Example 4

We have also determined the effect of the number of SNPs used in the LOH analysis of 97 ovarian cancer patients. When we used 336,958 SNPs we obtained a p-value of 0.000059. When number SNPs used in the analysis was decreased p-value became less and less significant until it finally became higher than 0.05 when number of SNPs dropped to 100. Results are shown in FIG. 10 (x-axis corresponds to the number of SNPs used in the LOH analysis while y-axis corresponds to the p-value).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. A method of classifying cancer comprising determining the amount of LOH in a sample containing cancer cells from said patient, wherein high LOH indicates a poor prognosis.

2. The method of claim 1, wherein said sample has high LOH if at least 35% of the genome of cells in said sample has LOH.

3. The method of claim 1, wherein said poor prognosis is an increased likelihood of shorter overall survival or shorter disease-free survival.

4. The method of claim 1, wherein said determining comprises genome-wide analysis.

5. The method of claim 4, wherein said determining comprises whole genome sequencing.

6. The method of claim 4, wherein said determining comprises genome-wide SNP analysis.

7. The method of claim 6, wherein at least 5,000 SNPs are analyzed.

8. The method of claim 1, wherein said determining comprises isolating nucleic acid from said sample and analyzing said nucleic acid to determine the amount of LOH.

9. The method of claim 1, wherein determining the amount of LOH comprises determining copy number in said sample using the analysis outlined in Example 2.

10. The method of claim 1, wherein said sample contains no more than 55% contamination with non-cancerous cells.

11. The method of claim 1, wherein said sample contains at least 5% contamination with non-cancerous cells.

12. The method of claim 1, wherein said sample is chosen from a frozen tissue sample and an FFPE sample.

13. The method of claim 1 further comprising determining LOH for a hotspot locus.

14. The method of claim 1, wherein the loci analyzed for LOH do not include any of the loci listed in Table A or Table B.

15. The method claim 1, wherein said patient has a cancer chosen from ovarian, breast, lung, prostate and colon.

16. A computer-implemented method of classifying cancer comprising:
accessing information on a patient contained in a computer-readable medium;
querying the data stored in said computer-readable medium to obtain LOH information for a sample from said patient;
determining whether said LOH information indicates said sample has a high amount of LOH; and
outputting or displaying that said patient has a poor prognosis if said sample has a high amount of LOH.

17. A method of treating a cancer patient comprising determining the amount of LOH in a sample containing cancer cells from said patient and administering, prescribing or recommending an aggressive treatment if said sample has a high amount of LOH.

18. A method comprising determining the genome-wide amount of LOH in a patient sample and determining whether a particular prognostic marker has LOH.

19. The method of claim 17, wherein the loci analyzed for LOH do not include any of the loci listed in Table A or Table B.

20. The method of claim 17, wherein either a high genome-wide amount of LOH or LOH in said prognostic marker indicates a poor prognosis.
